(19) United States
(12) Patent Application Publication
(10) Pub. No.: US 2024/0174765 A1
(43) Pub. Date: May 30, 2024

(54) ANTI- ENOLASE 1 (ENO1) ANTIBODY AND APPLICATIONS THEREOF

(71) Applicant: Academia Sinica, Taipei City (TW)

(72) Inventors: Han-Chung WU, Taipei City (TW); Hsin-Jung LI, New Taipei City (TW)

(73) Assignee: Academia Sinica, Taipei City (TW)

(21) Appl. No.: 18/281,977

(22) PCT Filed: Mar. 22, 2022
(86) PCT No.: PCT/US2022/021274

§ 371 Date: Sep. 14, 2023

Related U.S. Application Data
(60) Provisional application No. 63/164,137, filed on Mar. 22, 2021.

(51) Int. Cl. C07K 16/40; A61P 35/04; G01N 33/573; G01N 33/574

(52) U.S. Cl. CPC C07K 16/40; A61P 35/04; G01N 33/573; G01N 33/57492; G01N 2333/988

(57) ABSTRACT

The present invention relates to antibodies that bind to ENO1 and applications thereof. The applications encompass therapies and diagnostics of diseases or disorders associated with ENO1 activation and progression thereof using such antibodies. Specifically, the antibodies of the present invention bind to ENO1 on the surface of cancer cells and are useful in reducing cancer cell growth and metastasis and prolonging survival time. The antibodies of the present invention may also be used in detecting ENO1, diagnosis and prognosis of cancer and monitoring cancer progression. The present invention also provides a method for screening for a candidate agent for cancer therapy.

### RELATED APPLICATIONS

This application claims the benefit of U.S. provisional application No. 63/164,137, filed Mar. 22, 2021 under 35 U.S.C. § 119, the entire content of which is incorporated herein by reference.

### TECHNOLOGY FIELD

The present invention relates to antibodies that bind to enolase 1 (ENO1) and applications thereof. Specifically, the antibodies of the present invention bind to ENO1 on the surface of cancer cells and are effective in reducing cancer cell growth and metastasis and prolonging survival time. The antibodies of the present invention may also be used in detecting ENO1, diagnosis and prognosis of cancer and monitoring cancer progression. The present invention also provides a method for screening for a candidate agent for cancer therapy.

### BACKGROUND OF THE INVENTION

Enolase 1 (ENO1), also known as alpha-enolase, is a metabolic enzyme involved in the synthesis of pyruvate, and it also known to act as a plasminogen receptor and mediate the activation of plasmin and extracellular matrix degradation¹. Based on these mechanisms, ENO1 contributes to a wide variety of cellular and physiological processes, such as proteolysis, extracellular matrix remodeling, metabolism, growth control, tumor metastasis, and allergic response^{2,3}.

Overexpression of ENO1 mRNA or protein has been observed in many different tumor types, including lung, brain, breast, cervix, colon, gastric, head and neck, kidney, leukemia, liver, ovary, pancreas, prostate, skin and testicular cancers^{3,4}. In addition to its role as a glycolytic enzyme, ENO1 is expressed at the cell surface of most tumors, where it forms a multi-protein complex with urokinase-type plasminogen activator receptor (uPAR), integrins and certain cytoskeletal proteins, which are responsible for adhesion, migration and proliferation⁵. In tumors, ENO1 can modulate intravascular and pericellular fibrinolytic activity to promote cell migration and cancer metastasis⁶⁻⁸. In addition, the expression level of ENO1 may serve as a valuable diagnostic biomarker, as it is associated with patient outcomes, such as cancer survival and prognosis⁹⁻¹¹. For example, among patients with non-small-cell lung carcinoma (NSCLC), those with tumor cells expressing high levels of ENO1 have relatively poor disease-free and overall survival rates¹². Recent findings have shown that targeting ENO1 could be a novel approach to overcome drug resistance¹³. As such, tumor associated antigen (TAA)-ENO1 has become an intriguing target for cancer therapy.

Epithelial-to-mesenchymal transition (EMT) is a cellular program that naturally occurs in a broad range of tissue types and developmental stages¹⁴. However, it has become apparent that EMT is also important in the pathogenesis of many human diseases and cancers, based on its contributions to organ fibrosis^{15,16}, therapeutic resistance¹⁷, inflammatory response^{18,19}, immunosuppression^{20,21} and metastatic cell dissemination²². Furthermore, a growing body of evidence has shown that EMT is involved in many tumor metastasis processes, including local invasion of neoplastic cells at the primary tumor site, intravasation into blood vessels, circulation through the vasculature, extravasation into the parenchyma of distant tissues, and survival of micrometastatic deposits^{23,24}. In order to distinguish between cells on the extreme poles of the epithelial and mesenchymal axis, researchers typically examine certain cellular characteristics; epithelial cells display epithelial cell-cell junctions, apical-basal polarity and lack of motility, while mesenchymal cells exhibit heightened motility, invasiveness and resistance to apoptosis, along with spindle-like morphology that lacks apical-basal polarity²⁵. EMT is often detected as a morphological switch from an epithelial to a mesenchymal phenotype, including loss of epithelial cell markers (e.g., E-cadherin, α-catenin and γ-catenin) and gain of mesenchymal markers (e.g., vimentin, N-cadherin and fibronectin); this switch is executed by EMT-activating transcription factors (EMT-TFs), such as SLUG, SNAIL and TWIST^{23,26}. Therefore, EMT-TFs have become well known for their pleiotropic roles in cancer formation, growth and metastasis, which underlie their associations with poor clinical outcomes in many epithelial tumor types.

Wnt signaling is a critical pathway for embryonic development and adult cellular injury and repair processes. Upon Wnt ligand binding to its receptors, the capacity of the β-catenin destruction complex to phosphorylate cytosolic β-catenin is inhibited. Unphosphorylated β-catenin accumulates in the cytosol and translocates to the nucleus, where it activates expression of Wnt target genes. This genetic regulation requires integration with the TCF/LEF family of transcription factors and is important for controlling cellular proliferation, differentiation and survival in many contexts, including carcinoma progression^{27,28}. In addition to its role in the nucleus, E-cadherin forms a complex with β-catenin at the cell surface that facilitates the maintenance of cell-cell junctions²⁹. Similar to Wnt ligand biding, downregulation of E-cadherin can also increase levels of β-catenin in both cytoplasmic and nuclear compartments, stimulating cell growth and providing a mechanistic connection with EMT³⁰.

Another important signaling pathway governing EMT is stimulated by hepatocyte growth factor receptor (HGFR; also called c-Met). HGFR and its ligand HGF are involved in many normal and pathological biological processes, such as fetal development of liver, placenta, muscle and the nervous system^{31,32}. After birth, activation of the HGF-HGFR pathway appears to be involved in EMT³³, as well as hepatic, renal and epidermal regeneration³¹. Importantly, HGFR signaling also promotes tumor growth, invasion, drug resistance, angiogenesis and especially the generation and maintenance of cancer stem cells. Activation of the HGFR signaling pathway can occur by several different mechanisms, including increased HGF expression, enhanced HGFR protein expression, or by alteration of other factors or pathways affecting HGFR activation³⁴. Upon HGFR activation, phosphorylation of its carboxy-terminal tail creates a multifunctional docking site that recruits intracellular adapters and substrates (PI3K, Src and others)³⁵. Thus, several pathways involved in proliferation, survival, cell motility, invasion, and metastasis can be activated by this critical signaling pathway.

Our previous findings indicated that ENO1-targeting liposomes loaded with anti-cancer drugs have tremendous potential for application as a targeted drug delivery system for cancer therapy³⁶. However, the detailed signaling mechanism regarding how ENO1 participates in carcinogenesis was unclear, and so far, no anti-ENO1 drug candidates have progressed to clinical trials. In this filed, there is still a need for efficacious anti-ENO1 antibodies.

### SUMMARY OF THE INVENTION

Disclosed here are antibodies that bind to ENO1 and applications thereof. Cells that can be targeted by the antibodies include cancer cells. The applications include without limitation cancer therapy and diagnostic and prognostic of cancer. Specifically, the antibodies are useful in reducing cancer cell growth and metastasis and prolonging survival time. The antibodies may also be used in detecting ENO1 and diagnostic and prognostic of cancer and monitoring cancer progression in a patient afflicted with cancer.

Therefore, in one aspect, the present invention provides an anti-ENO1 antibody or antigen-binding fragment thereof, which comprises
(a) a heavy chain variable region (VH) which comprises a heavy chain complementary determining region 1 (HC CDR1) comprising the amino acid sequence of SEQ ID NO: 2, a heavy chain complementary determining region 2 (HC CDR2) comprising the amino acid sequence of SEQ ID NO: 4, and a heavy chain complementary determining region 3 (HC CDR3) comprising the amino acid sequence of SEQ ID NO: 6; and
(b) a light chain variable region (VL) which comprises a light chain complementary determining region 1 (LC CDR1) comprising the amino acid sequence of SEQ ID NO: 9, a light chain complementary determining region (LC CDR2) comprising the amino acid sequence of SEQ ID NO: 11, and a light chain complementary determining region 3 (LC CDR3) comprising the amino acid sequence of SEQ ID NO: 13.

In some embodiments, the VH comprises the amino acid sequence of SEQ ID NO: 15, and/or the VL comprises the amino acid sequence of SEQ ID NO: 16.

Also provided are a recombinant nucleic acid comprising a nucleotide sequence encoding an anti-ENO1 antibody or antigen-binding fragment as described herein and a host cell comprising the recombinant nucleic acid.

Further provided is a composition comprising an anti-ENO1 antibody or antigen-binding fragment thereof as described herein and a physiologically acceptable carrier.

In another aspect, the present invention provides a method for inhibiting ENO1-downstream signaling and/or treating a disease or disorder associated with activation of ENO1-downstream signaling by administering to a subject in need thereof an effective amount of an anti-ENO1 antibody or antigen-binding fragment thereof or a composition comprising the same as described herein.

In some embodiments, the disease or disorder is a cancer and a metastasis thereof.

In certain embodiments, the cancer is selected from the group consisting of lung cancer, brain cancer, breast cancer, cervical cancer, colon cancer, gastric cancer, head and neck cancer, kidney cancer, leukemia, liver cancer, ovarian cancer, pancreatic cancer, prostate cancer, skin cancer and testicular cancer. In one particular example, the cancer is lung cancer.

In particular embodiments, the antibody or antigen-binding fragment thereof as described herein is administered in an amount effective to inhibit tumor growth and metastasis in the subject in need thereof

In particular embodiments, the antibody or antigen-binding fragment thereof as described herein is administered in an amount effective in prolonging survival time in the subject in need thereof.

In a further aspect, the present invention provides a method for deterring ENO1, comprising
(i) contacting an antibody or antigen-binding fragment thereof as described herein with a cell of a subject in need; and
(ii) detecting binding of the antibody or antigen-binding fragment thereof to the cell.

In some embodiments, the method is performed in vitro where the cell is present in a biological sample obtained from the subject. In some embodiments, the method is performed in vivo where the cell is present in the subject.

In some embodiments, the subject is at risk for, or suspected of having a cancer.

In some embodiments, the subject is afflicted with a cancer.

In some embodiments, the method for detecting ENO1 as described herein further comprises comparing the results of the detection with a reference level and determining prognosis for the subject afflicted with cancer based on the results of the comparison, wherein an elevated level of ENO1 is indicative of a negative prognosis.

In particular embodiments, the negative prognosis is selected from the group consisting of a reduced survival rate, an increased tumor growth, an increased risk of metastasis, an increased risk of relapse, and any combination thereof.

In still a further aspect, the present invention provides a method for monitoring cancer progression in a patient afflicted with cancer comprising
(a) providing a first biological sample from the patient at a first time point;
(b) providing a second biological sample from the patient at a second time point, which is later than the first time point;
(c) detecting the levels of ENO1 in the first and second biological samples, wherein the detection is performed by an immunoassay with an anti-ENO1 antibody or antigen-binding fragment thereof as described herein; and
(d) determining cancer progression in the patient based on the levels of ENO1 in the first and second biological samples, wherein an elevated level of ENO1 in the second biological sample as compared to that in the first biological sample is indicative of cancer progression.

The present invention also describes an anti-ENO1 antibody or antigen-binding fragment thereof or a composition comprising the same as described herein for use in inhibiting ENO1-downstream signaling and/or treating a disease or disorder associated with activation of ENO1-downstream signaling. Further disclosed is use of an anti-ENO1 antibody or antigen-binding fragment thereof or a composition comprising the same as described herein for manufacturing a medicament for inhibiting ENO1-downstream signaling and/or treating a disease or disorder associated with activation of ENO1-downstream signaling. Examples of such disease or disorder are cancer and metastasis thereof. In certain embodiments, the cancer is selected from the group consisting of lung cancer, brain cancer, breast cancer, cervical cancer, colon cancer, gastric cancer, head and neck cancer, kidney cancer, leukemia, liver cancer, ovarian cancer, pancreatic cancer, prostate cancer, skin cancer and testicular cancer. In one particular example, the cancer is lung cancer.

The present invention also disclosed an anti-ENO1 antibody or antigen-binding fragment thereof or a composition comprising the same as described herein for use in detecting ENO1, diagnosis and prognosis of cancer and/or monitoring cancer progression in a patient afflicted with cancer. Further disclosed is use of an anti-ENO1 antibody or antigen-binding fragment thereof or a composition comprising the same as described herein for manufacturing a kit for detecting ENO1, diagnosis and prognosis of cancer and/or monitoring cancer progression in a patient afflicted with cancer. The kit comprises an anti-ENO1 antibody or antigen-binding fragment thereof or a composition comprising the same as described herein and instructions for using the kit to detect ENO1. Specifically, the kit is an immunoassay kit. In certain embodiments, the cancer is selected from the group consisting of lung cancer, brain cancer, breast cancer, cervical cancer, colon cancer, gastric cancer, head and neck cancer, kidney cancer, leukemia, liver cancer, ovarian cancer, pancreatic cancer, prostate cancer, skin cancer and testicular cancer. In one particular example, the cancer is lung cancer.

The present invention also provides a method for screening for a candidate agent for cancer therapy. In particular, the method of the invention comprising (i) contacting an agent with a cancer cell expressing ENO1 on the cell surface of the cancer cell; (ii) detecting binding of the agent with ENO1 on the cell surface of the cancer cell and/or measuring the levels of one or more ENO1-downstream signaling events/components in the cancer cell; and (iii) determining if the agent is a candidate anti-cancer based on the results of the detection and/or measurement.

The details of one or more embodiments of the invention are set forth in the description below. Other features or advantages of the present invention will be apparent from the following detailed description of several embodiments, and also from the appending claims.

### BRIEF DESCRIPTION OF THE DRAWINGS

The foregoing summary, as well as the following detailed description of the invention, will be better understood when read in conjunction with the appended drawings. For the purpose of illustrating the invention, there are shown in the drawings embodiments which are presently preferred. It should be understood, however, that the invention is not limited to the precise arrangements and instrumentalities shown.

In the drawings:

FIGS. 1A to 1I. ENO1 is highly expressed in metastatic cancer cell lines and is associated with worse overall survival of lung cancer patients. (FIG. 1A) ENO1 expression was analyzed by Western blotting in NHBE, NL-20 and human lung cancer cell lines using an anti-ENO1 antibody. α-Tubulin served as an internal control. (FIG. 1B) The binding of chimeric ENO1 mAb (chENO1) or Normal human IgG (NHIgG) to live NHBE, NL-20 or CL1-5 cells was evaluated by flow cytometry using phycoerythrin (PE)-conjugated goat anti-mouse IgG. X axis, intensity of PE fluorescence; Y axis, number of events. (FIG. 1C) Immunogold labelling of ENO1 in CL1-5 cells. Cells were fixed with 4% formaldehyde and processed for cryosectioning. Sections were blocked with BSA, followed by a labeling for ENO1 with chENO1-22 (40 μg/ml) or NHIgG. After fixation, the samples were stained and examined on a transmission electron microscope. (FIG. 1D) The mRNA expression of ENO1 in NSCLC tumor tissues (T) and adjacent noncancerous tissues (N) was analyzed from the TCGA dataset. (FIG. 1E) Representative images show ENO1 immunohistochemical staining in samples across the full spectrum of lung cancer stages (original magnification, 40×; scale bar=200 μm). (FIG. 1F) Immunohistochemical staining data were expressed as the area percentage positive staining of ENO1. The median for each group is indicated by a horizontal bar. (FIG. 1G) Overall survival analysis of the protein expression of ENO1 based on the prognostic information of patients with lung cancer from tissue microarray data. OS was analyzed using the Kaplan-Meier plotter (KM plotter) (FIG. 1H) and TCGA dataset (I). The hazard ratio (HR) with 95% confidence intervals and log rank P values from the webpage are provided. **P<0.01 and ***P<0.001.

FIGS. 2A to 2F. Identification of the molecular functions of ENO1 in lung cancer cells in vitro. (FIG. 2A) Expression of ENO1 in CL1-5 or CL-141 cells infected with lentiviruses harboring control shRNA (shLacZ) or ENO1 shRNA (shENO1-1 and shENO1-2) was analyzed by Western blot. α-Tubulin served as an internal control. Cell proliferation (WST-1) assays were performed using ENO1-knockdown CL1-5 or CL-141 cells (FIG. 2B) and CL1-0) cells (FIG. 2E). The absorbance values are presented as mean±SEM from four independent experiments. Matrigel invasion assays were performed using ENO1-knockdown CL1-5 or CL-141 cells (FIG. 2C) and CL1-0) cells (FIG. 2F). The invading cells were counted and the results are presented as mean±SEM from three independent experiments. (*P<0.5 and ***P<0.001, Student's t-test) (FIG. 2D) Expression of ENO1 in CL1-0) cells transfected with AS2-ENO1-FLAG or the empty vector was analyzed by Western blot using an anti-FLAG antibody.

FIGS. 3A to 3E. ENO1 knockdown inhibits tumor growth and lung tumor metastasis in mouse xenograft models. CL1-5-Luci cells were infected with lentiviruses harboring control shLacZ or shENO1 (each also expressing luciferase) and transplanted into the lung parenchyma (1×10⁵ cells) (FIG. 3A and FIG. 3B) or injected into the tail vein (1×10⁶ cells) of NOD-SCID mice. (FIG. 3C, FIG. 3D, FIG. 3E). Tumor formation in the lungs was monitored by bioluminescence imaging. (FIG. 3E) The number of tumor nodules was counted after hematoxylin and eosin staining of lungs excised from the mice shown in (FIG. 3C). **P<0.05; ***P<0.001.

FIGS. 4A to 4G. ENO1 promotes EMT by regulating SLUG stability. (FIG. 4A) The morphology of CL1-5 cells infected with lentiviruses harboring control shLacZ or shENO1. (FIG. 4B) The expression of EMT markers or regulators was detected by Western blotting in CL1-5 cells infected with shLacZ or shENO1 (Left) or CL1-0) cells infected with vector or ENO1-FLAG (Right). (FIG. 4C) The expression of EMT markers or regulators was detected by qRT-PCR in CL1-5 cells infected with shLacZ or shENO1. (FIG. 4D) The protein expression of these genes was analyzed in ENO1-knockdown cells by treating with or without 10 mM MG132 (proteasome inhibitor) for 6 h, followed by Western blotting. (FIG. 4E) CL1-5 cells were infected with lentiviruses harboring shLacZ or shENO1. Twenty-four hours after transfection, the cells were treated with 10 mM MG132 for 6 h before cell collection. The lysates were subjected to immunoprecipitation using anti-SLUG antibody (Left panel) and input (Right panel). Western blotting was performed with the indicated antibodies to detect protein interactions. (FIG. 4F) Stability of SLUG protein in CL1-5 cells infected with shLacZ or shENO1. Cells were treated with cyclohexamide (CHX) 100 μg at the indicated intervals and subjected to Western blotting analysis. (FIG. 4G) Quantification of SLUG half-life in indicated groups. **P<0.05; ***P<0.001.

FIGS. 5A to 5G. ENO1 stabilizes SLUG by decreasing GSK3β activity via activation of HGFR-Src-PI3K-AKT and Wnt-β-catenin signaling. (FIG. 5A) Protein expression was analyzed by Western blotting in ENO1-knockdown cells (shENO1-1) after treatment with or without 2 μM GSK3β inhibitor (BIO) for 24 h. (FIG. 5B) One set of cells was transfected with the TOP reporter, CMV: Renilla luciferase; a second set of cells was transfected with the FOP reporter, CMV: Renilla luciferase as indicated. The TOP and FOP reporters are as follows: β-catenin-responsive “T cell factor (TCF) element driving luciferase” (TOP); and non-β-catenin-responsive mutated element also driving luciferase (FOP). The ratio of signals from these two reporters, each normalized to Renilla luciferase as internal control, reflects β-catenin-specific canonical Wnt signaling. After 72 h, Wnt signaling was measured in ENO1-knockdown CL1-5 cells or control cells. (FIG. 5C) Confocal immunofluorescence assays were used to detect B-catenin expression in CL1-5 cells. A 63× objective was used to observe the samples. Green fluorescence represents β-catenin, and blue fluorescence is the DAPI stain. The lower panel shows quantification of the mean intensity for nuclear SLUG. (FIG. 5D) Matrigel invasion assays were performed on CL1-5 cells expressing indicated plasmid combinations. (FIG. 5E) TCGA data analysis (RNA-Sequence data of lung adenocarcinoma) of overall survival for NSCLC patients with ENO1 low/SLUG low; ENO1 high/SLUG low; ENO1 low/SLUG high, and ENO1 high/SLUG high. Patients were split according to the median. (FIG. 5F) Protein levels were detected by Western blotting in CL1-5 cells infected with shLacZ or ENO1 shRNA or ENO1 shRNA cells were treated with recombinant ENO1 for 30 min (1 μg/mL). P: phosphorylated protein levels, t: total protein levels. (FIG. 5G) Matrigel invasion assays were performed using CL1-5 cells infected with shLacZ or ENO1 shRNA or ENO1 shRNA cells were treated with recombinant ENO1 for 30 min (1 or 2 μg/mL). The invading cells were counted and the results are presented as mean±SEM from three independent experiments. **P<0.01; ***P<0.001.

FIGS. 6A to 6G. ENO1 associates with HGFR and is essential for HGFR. (FIG. 6A) CL1-5 cells infected with shLacZ or ENO1 shRNA cells were stimulated with vehicle or human HGF for 10 min followed by immunoblotting. (FIG. 6B) GST pull-down assay. GST-agarose bead bound GST-ENO1 or GST proteins were incubated with purified His-HGFR. After washing, the bead-bound proteins and 5% input His-HGFR protein were released by boiling with SDS-PAGE loading buffer. Samples were then subjected to Western blotting with anti-His or ENO1 antibodies. (FIG. 6C) Membrane proteins were extracted and IP of affinity cross-linked ENO1 bound to HGFR from CL1-5 cells. (FIG. 6D) Immunoprecipitates were prepared from CL1-5 cells after infection with FLAG-tagged ENO1. Cell lysates were immunoprecipitated with anti-ENO1 (IP: ENO1) or anti-HGFR (IP: HGFR) antibodies and blotted with anti-HGFR and anti-ENO1 antibodies. (FIG. 6E) Immunoprecipitates were prepared from CL1-5 cells. Cell lysates were immunoprecipitated with anti-ENO1 (IP: ENO1) or anti-HGFR (IP: HGFR) antibodies and blotted with anti-HGFR and anti-ENO1 antibodies. (FIG. 6F) Upper panel: schematic diagram of mapping the HGFR structural domains. Different Myc-tagged-HGFR constructs or an empty vector for 72 h were immunoprecipitated with anti-Myc antibodies and fractionated by SDS-polyacrylamide gel electrophoresis (PAGE). Immunoblots were probed with anti-FLAG or anti-Myc antibodies. (FIG. 6G) CL1-5 cells infected with shLacZ or ENO1 shRNA cells were treated HGFR inhibitor for 24 h and stimulated with hHGF for 10 min followed by immunoblotting.

FIGS. 7A to 7I. chENO1-22 antibody decreases cancer cell proliferation and invasion in vitro by inhibiting ENO1-HGFR axis-mediated downstream signaling to promote SLUG protein degradation. Quantitation of the binding of anti-chENO1-22 to live CL1-5 cells infected with lentiviruses harboring LacZ or ENO1 shRNA (FIG. 7A) or CL1-0) cells transfected with AS2-ENO1-FLAG or the empty vector (FIG. 7B) is evaluated by flow cytometry using phycoerythrin (PE)-conjugated goat anti-human IgG. (FIG. 6C) Cell proliferation (WST-1) assays were performed using CL1-5 cells treated with two mouse mAbs against ENO1. The concentration of antibodies was 10 μg/ml, and the OD values were measured 8, 24, 48 or 72 h after antibody treatment. (FIG. 6D) Matrigel invasion assays were performed using CL1-5 cells treated with mouse mAbs against ENO1. The invading cells were quantified, and the results are presented as the mean±SEM from three independent experiments (*P<0.05, Student's t-test). Lower pictures show fields of invasive cells in the matrigel invasion assay. (FIG. 6E) Protein levels were detected by Western blotting of CL1-5 cells treated with NHIgG or chENO1-22. (FIG. 6F) CL1-5 cells were treated with NHIgG or chENO1-22 for 4 h and then stimulated with vehicle or hHGF for 10 min followed by Western blotting. (FIG. 6G) CL1-5 cells were treated with NHIgG or chENO1-22 for 4 h and then immunoprecipitated with anti-ENO1 (IP: ENO1) or anti-HGFR (IP: HGFR) antibodies followed by Western blotting. (FIG. 6H) CL1-5 cells were treated with 10 mM MG132 and chENO1-22 for 6 h before cell collection and subsequent Western blotting. (FIG. 6I) Stability of Slug protein in CL1-5 cells treated with NHIgG or chENO1-22. Cells were treated with cyclohexamide (CHX) 100 μg at the indicated intervals and subjected to Western blotting analysis. Lower graph shows quantification of SLUG half-life in indicated groups. *P<0.05; **P<0.01; ***P<0.001.

FIGS. 8A to 8D. chENO1-22 antibody decreases lung cancer cell metastasis in vivo. (FIG. 8A) One million CL1-5-Luci cells were i.v. injected into NOD-SCID mice and the normal human IgG control or chENO1-22 antibodies were i.v. injected two times a week for a total of eight injections. Weekly bioluminescence imaging was performed to monitor the growth of the i.v.-injected tumor cells (Median survival day: Log-rank, p=0.0019). (FIG. 8B) Tumor formation in the lungs was monitored by bioluminescence imaging. (FIG. 8C) Kaplan-Meier curves were generated to analyze survival. (FIG. 8D) A working model depicts ENO1 promotion of cell invasion through regulation of the HGFR-Src-PI3K-AKT-GSK3β-SLUG and HGFR-WNT-GSK3β-SLUG axes. (a) ENO1 increases the phosphorylation of HGFR and Wnt co-receptor LPR5/6 and decreases GSK-3β activity to enhance SLUG stabilization and suppress E-cadherin expression. Ultimately, cancer cell invasion capacity is enhanced. (b) The chENO1-22 mAb inhibits cancer cell invasion by blocking ENO1-HGFR axis-mediated downstream signaling to promote SLUG protein ubiquitination and degradation.

FIGS. 9A to 9D. To screen the binding activity and specificity of anti-ENO1 mAbs by hybridoma technology. Binding activity of four anti-ENO1 mAbs to CL1-5 cells is analyzed by cellular ELISA (FIG. 9A) and flow cytometry on live cells using phycoerythrin (PE)-conjugated goat anti-mouse IgG as a secondary antibody (FIG. 9B). X axis, intensity of PE fluorescence; Y axis, number of events. Quantitation of the binding of anti-chENO1-22 to live CL1-5 cells (FIG. 9C) or CL1-5 cells infected with lentiviruses harboring LacZ or ENO1 shRNA (FIG. 9D) is evaluated by flow cytometry using phycoerythrin (PE)-conjugated goat anti-human IgG.

FIG. 10. ENO1 expression is associated with worse cancer stages of lung cancer patients. Immunohistochemical staining data were expressed as the intensity percentage of positive staining of ENO1. ENO1 expression was classified into 1 of 4 categories according to the ENO1 staining intensity: 3, intense expression; 2, moderate expression; 1, faint or equivocal expression; and 0, no expression.

FIG. 11. LRP5 and LRP6 were essential for ENO1-induced SLUG and β-catenin stabilization. CL1-5 cells infected with shLacZ or ENO1 shRNA or LRP5/6 shRNA were stimulated with vehicle or hHGF for 10 min followed by Western blotting.

FIG. 12. ChENO1-22 antibody decreases lung cancer cell metastasis in vivo. The mice were dosed with 10 or 20 mg/kg chENO1 and body weight of mice treated are shown.

FIG. 13. Amino acid sequences of VH and VL of chENO1-22 antibody of the present invention.

### DETAILED DESCRIPTION OF THE INVENTION

The following description is merely intended to illustrate various embodiments of the invention. As such, specific embodiments or modifications discussed herein are not to be construed as limitations to the scope of the invention. It will be apparent to one skilled in the art that various changes or equivalents may be made without departing from the scope of the invention.

### I. Definitions

In order to provide a clear and ready understanding of the present invention, certain terms are first defined. Additional definitions are set forth throughout the detailed description. Unless defined otherwise, all technical and scientific terms used herein have the same meanings as is commonly understood by one of skill in the art to which this invention belongs.

As used herein, the singular forms “a”, “an”, and “the” include plural referents unless the context clearly dictates otherwise. Thus, for example, reference to “a component” includes a plurality of such components and equivalents thereof known to those skilled in the art.

The term “comprise” or “comprising” is generally used in the sense of include/including which means permitting the presence of one or more features, ingredients or components. The term “comprise” or “comprising” encompasses the term “consists” or “consisting of.”

As used herein, the term “polypeptide” refers to a polymer composed of amino acid residues linked via peptide bonds. The term “protein” typically refers to relatively large polypeptides. The term “peptide” typically refers to relatively short polypeptides (e.g., containing up to 100, 90, 70, 50, 30, 20 or 10 amino acid residues).

As used herein, the term “approximately” or “about” refers to a degree of acceptable deviation that will be understood by persons of ordinary skill in the art, which may vary to some extent depending on the context in which it is used. Specifically, “approximately” or “about” may mean a numeric value having a range of ±10% or ±5% or ±3% around the cited value.

As used herein, the term “substantially identical” refers to two sequences having 80% or more, preferably 85% or more, more preferably 90% or more, even more preferably 95% or more homology.

As used herein, the term “antibody” (interchangeably used in plural form, antibodies) means an immunoglobulin molecule having the ability to specifically bind to a particular target antigenic molecule. As used herein, the term “antibody” includes not only intact (i.e. full-length) antibody molecules but also antigen-binding fragments thereof retaining antigen binding ability e.g. Fab, Fab′, F(ab′)2 and Fv. Such fragments are also well known in the art and are regularly employed both in vitro and in vivo. The term “antibody” also includes chimeric antibodies, humanized antibodies, human antibodies, diabodies, linear antibodies, single chain antibodies, multispecific antibodies (e.g., bispecific antibodies) and any other modified configuration of the immunoglobulin molecule that comprises an antigen recognition site of the required specificity, including amino acid sequence variants of antibodies, glycosylation variants of antibodies, and covalently modified antibodies.

An intact or complete antibody comprises two heavy chains and two light chains. Each heavy chain contains a variable region (V_{H}) and a first, second and third constant regions (C_{H}1, C_{H}2 and C_{H}3); and each light chain contains a variable region (V_{L}) and a constant region (C_{L}). The antibody has a “Y” shape, with the stem of the Y consisting of the second and third constant regions of two heavy chains bound together via disulfide bonding. Each arm of the Y includes the variable region and first constant region of a single heavy chain bound to the variable and constant regions of a single light chain. The variable regions of the light chains and those of heavy chains are responsible for antigen binding. The variables regions in both chains are responsible for antigen binding generally, each of which contain three highly variable regions, called the complementarity determining regions (CDRs); namely, heavy (H) chain CDRs including HC CDR1, HC CDR2, HC CDR3 and light (L) chain CDRs including LC CDR1, LC CDR2, and LC CDR3. The three CDRs are franked by framework regions (FR1, FR2, FR3, and FR4), which are more highly conserved than the CDRs and form a scaffold to support the hypervariable regions. The constant regions of the heavy and light chains are not responsible for antigen binding, but involved in various effector functions. Depending on the antibody amino acid sequence of the constant domain of its heavy chains, immunoglobulins can be assigned to different classes. There are five major classes of immunoglobulins: IgA, IgD, IgE, IgG, and IgM. The heavy-chain constant domains that correspond to the different classes of immunoglobulins are called alpha, delta, epsilon, gamma, and mu, respectively.

As used herein, the term “antigen-binding fragment” or “antigen-binding domain” refers to a portion or region of an intact antibody molecule that is responsible for antigen binding. An antigen-binding fragment is capable of binding to the same antigen to which the parent antibody binds. Examples of antigen-binding fragments include, but are not limited to: (i) a Fab fragment, which can be a monovalent fragment composed of a V_{H}-C_{H}1 chain and a V_{L}-C_{L} chain; (ii) a F(ab′)2 fragment which can be a bivalent fragment composed of two Fab fragments linked by a disulfide bridge at the hinge region; (iii) a Fv fragment, composed of the V_{H} and V_{L} domains of an antibody molecule associated together by noncovalent interaction; (iv) a single chain Fv (scFv), which can be a single polypeptide chain composed of a V_{H} domain and a V_{L} domain via a peptide linker; and (v) a (scFv)₂, which can contain two V_{H} domains linked by a peptide linker and two V_{L} domains, which are associated with the two V_{H} domains via disulfide bridges.

As used herein, the term “chimeric antibody” refers to an antibody containing polypeptides from different sources, e.g., different species. In some embodiments, in chimeric antibodies, the variable region of both light and heavy chains may mimic the variable region of antibodies derived from one species of mammal (e.g., a non-human mammal such as mouse, rabbit and rat), while the constant region may be homologous to the sequences in antibodies derived from another mammal such as a human.

As used herein, the term “humanized antibody” refers to an antibody comprising a framework region originated from a human antibody and one or more CDRs from a non-human (usually a mouse or rat) immunoglobulin.

As used herein, the term “human antibody” refers to an antibody in which essentially the entire sequences of the light chain and heavy chain sequences, including the complementary determining regions (CDRs), are from human genes. In some circumstances, the human antibodies may include one or more amino acid residues not encoded by human germline immunoglobulin sequences e.g. by mutations in one or more of the CDRs, or in one or more of the FRs, such as to, for example, decrease possible immunogenicity, increase affinity, and eliminate cysteines that might cause undesirable folding, etc.

As used herein, the term “specific binds” or “specifically binding” refers to a non-random binding reaction between two molecules, such as the binding of the antibody to an epitope of its target antigen. An antibody that “specifically binds” to a target antigen or an epitope is a term well understood in the art, and methods to determine such specific binding are also well known in the art. An antibody “specifically binds” to a target antigen if it binds with greater affinity/avidity, more readily, and/or greater duration than it binds to other substances. In other words, it is also understood by reading this definition that, for example, an antibody that specifically binds to a first target antigen may or may not specifically or preferentially bind to a second target antigen. As such, “specific binding” or “preferential binding” does not necessarily require (although it can include) exclusive binding. Generally, the affinity of the binding can be defined in terms of a dissociation constant (K_{D}). Typically, specifically binding when used with respect to an antibody can refer to an antibody that specifically binds to (recognize) its target with an KD value less than about 10^{˜7} M, such as about 10^{˜8} M or less, such as about 10^{˜9} M or less, about 10^{˜10} M or less, about 10^{˜n} M or less, about 10″¹² M or less, or even less, and binds to the specific target with an affinity corresponding to a K_{D} that is at least ten-fold lower than its affinity for binding to a non-specific antigen (such as BSA or casein), such as at least 100 fold lower, e.g. at least 1,000 fold lower or at least 10,000 fold lower.

As used herein, the term “nucleic acid” or “polynucleotide” can refer to a polymer composed of nucleotide units. Polynucleotides include naturally occurring nucleic acids, such as deoxyribonucleic acid (“DNA”) and ribonucleic acid (“RNA”) as well as nucleic acid analogs including those which have non-naturally occurring nucleotides. Polynucleotides can be synthesized, for example, using an automated DNA synthesizer. It will be understood that when a nucleotide sequence is represented by a DNA sequence (i.e., A, T, G, C), this also includes an RNA sequence (i.e., A, U, G, C) in which “U” replaces “T.” The term “cDNA” refers to a DNA that is complementary or identical to an mRNA, in either single stranded or double stranded form.

As used herein, the term “complementary” refers to the topological compatibility or matching together of interacting surfaces of two polynucleotides. A first polynucleotide is complementary to a second polynucleotide when the nucleotide sequence of the first polynucleotide is identical to the nucleotide sequence of the polynucleotide binding partner of the second polynucleotide. Thus, the polynucleotide whose sequence 5′-ATATC-3′ is complementary to a polynucleotide whose sequence is 5′-GATAT-3′.”

As used herein, the term “encoding” refers to the natural property of specific sequences of nucleotides in a polynucleotide (e.g., a gene, a cDNA, or an mRNA) to serve as templates for synthesis of other polymers and macromolecules in biological processes having either a given sequence of RNA transcripts (i.e., rRNA, tRNA and mRNA) or a given sequence of amino acids and the biological properties resulting therefrom. Therefore, a gene encodes a protein if transcription and translation of mRNA produced by that gene produces the protein in a cell or other biological system. It is understood by a skilled person that numerous different polynucleotides and nucleic acids can encode the same polypeptide as a result of the degeneracy of the genetic code. It is also understood that skilled persons may, using routine techniques, make nucleotide substitutions that do not affect the polypeptide sequence encoded by the polynucleotides described there to reflect the codon usage of any particular host organism in which the polypeptides are to be expressed. Therefore, unless otherwise specified, a “nucleotide sequence encoding an amino acid sequence” encompasses all nucleotide sequences that are degenerate versions of each other and that encode the same amino acid sequence.

As used herein, the term “recombinant nucleic acid” refers to a polynucleotide or nucleic acid having sequences that are not naturally joined together. A recombinant nucleic acid may be present in the form of a vector. “Vectors” may contain a given nucleotide sequence of interest and a regulatory sequence. Vectors may be used for expressing the given nucleotide sequence (expression vector) or maintaining the given nucleotide sequence for replicating it, manipulating it or transferring it between different locations (e.g., between different organisms). Vectors can be introduced into a suitable host cell for the above-described purposes. A “recombinant cell” refers to a host cell that has had introduced into it a recombinant nucleic acid. “A transformed cell” mean a cell into which has been introduced, by means of recombinant DNA techniques, a DNA molecule encoding a protein of interest.

Vectors may be of various types, including plasmids, cosmids, episomes, fosmids, artificial chromosomes, phages, viral vectors, etc. Typically, in vectors, the given nucleotide sequence is operatively linked to the regulatory sequence such that when the vectors are introduced into a host cell, the given nucleotide sequence can be expressed in the host cell under the control of the regulatory sequence. The regulatory sequence may comprise, for example and without limitation, a promoter sequence (e.g., the cytomegalovirus (CMV) promoter, simian virus 40 (SV40) early promoter, T7 promoter, and alcohol oxidase gene (AOX1) promoter), a start codon, a replication origin, enhancers, a secretion signal sequence (e.g., α-mating factor signal), a stop codon, and other control sequence (e.g., Shine-Dalgarno sequences and termination sequences). Preferably, vectors may further contain a marker sequence (e.g., an antibiotic resistant marker sequence) for the subsequent screening/selection procedure. For purpose of protein production, in vectors, the given nucleotide sequence of interest may be connected to another nucleotide sequence other than the above-mentioned regulatory sequence such that a fused polypeptide is produced and beneficial to the subsequent purification procedure. Said fused polypeptide includes a tag for purpose of purification e.g. a His-tag.

As used herein, the term “treatment” refers to the application or administration of one or more active agents to a subject afflicted with a disorder, a symptom or condition of the disorder, or a progression of the disorder, with the purpose to cure, heal, alleviate, relieve, alter, remedy, ameliorate, improve, or affect the disorder, the symptom or condition of the disorder, the disabilities induced by the disorder, or the progression or predisposition of the disorder.

As used herein, the term “diagnosis” as used herein generally includes determination as to whether a subject is likely affected by a given disease, disorder or dysfunction. The skilled persons often make a diagnosis on the basis of one or more diagnostic indicators, i.e., a marker, the presence, absence, or amount of which is indicative of the presence or absence of the disease, disorder or dysfunction. It will be understood in the art that diagnosis does not mean determining the presence or absence of a particular disease with 100% accuracy, but rather an increased likelihood of the presence of certain disease in a subject.

As used herein, the term “prognosis” as used herein generally refers to a prediction of the probable course and outcome of a clinical condition or disease. A prognosis of a patient is usually made by evaluating factors or symptoms of a disease that are indicative of a favorable or unfavorable course or outcome of the disease. It is understood that the term “prognosis” does not necessarily refer to the ability to predict the course or outcome of a condition with 100% accuracy. Instead, the skilled persons will understand that the term “prognosis” refers to an increased probability that a certain course or outcome will occur: that is, that a course or outcome is more likely to occur in a patient exhibiting a given condition, when compared to those individuals not exhibiting the condition. It would be understandable that a positive prognosis typically refers to a beneficial clinical outcome or outlook, such as long-term survival without recurrence of the subject's cancerous conditions, whereas a negative (poor) prognosis typically refers to a negative clinical outcome or outlook, such as cancer recurrence or progression.

### II. Antibodies Against ENO1

The present invention is based, at least in part, on the generation of an anti-ENO1 monoclonal antibody (mAb-22) which can reliably detect ENO1 on the surface of cancer cells. The amino acid sequences of the heavy chain variable region (V_{H}) and light chain variable region (V_{L}), and their complementary determining regions (HC CDR1, HC CDR2 and HC CDR3) (LC CDR1, LC CDR2 and LC CDR3) of mAb-22 are as shown in Table 1 below: See Example 2.5. The anti-ENO1 antibody of the present invention includes mAb-22 and its functional variant.

In some embodiments, the anti-ENO1 antibody of the present invention is a functional variant of mAb-22 which is characterized in comprising (a) a VH comprising HC CDR1 of SEQ ID NO: 2, HC CDR2 of SEQ ID NO: 4, and HC CDR3 of SEQ ID NO: 6: and (b) a V_{L} comprising LC CDR1 of SEQ ID NO: 9, LC CDR2 of SEQ ID NO: 11, and HC CDR3 of SEQ ID NO: 13, or an antigen-binding fragment thereof.

In some embodiments, the anti-ENO1 antibody of the present invention, having (a) a VH comprising HC CDR1 of SEQ ID NO: 2, HC CDR2 of SEQ ID NO: 4, and HC CDR3 of SEQ ID NO: 6; and (b) a V_{L} comprising LC CDR1 of SEQ ID NO: 9, LC CDR2 of SEQ ID NO: 11, and HC CDR3 of SEQ ID NO: 13, can comprise a V_{H} comprising SEQ ID NO: 15 or an amino acid sequence substantially identical thereto and a V_{L} comprising SEQ ID NO: 16 or an amino acid sequence substantially identical thereto. Specifically, the anti-ENO1 antibody of the present invention includes a V_{H} comprising an amino acid sequence has at least 80% (e.g. 82%, 84%, 85%, 86%, 88%, 90%, 92%, 94%, 95%, 96%, 98%, or 99%) identity to SEQ ID NO: 15, and a V_{L} comprising an amino acid sequence has at least 80% (e.g. 82%, 84%, 85%, 86%, 88%, 90%, 92%, 94%, 95%, 96%, 98%, or 99%) identity to SEQ ID NO: 16. The anti-ENO1 antibody of the present invention also includes any recombinantly (engineered)-derived antibody encoded by the polynucleotide sequence encoding the relevant V_{H} or V_{L} amino acid sequences as described herein.

The term “substantially identical” can mean that the relevant amino acid sequences (e.g., in FRs, CDRs, V_{H}, or V_{L}) of a variant differ insubstantially as compared with a reference antibody such that the variant has substantially similar binding activities (e.g., affinity, specificity, or both) and bioactivities relative to the reference antibody. Such a variant may include minor amino acid changes. It is understandable that a polypeptide may have a limited number of changes or modifications that may be made within a certain portion of the polypeptide irrelevant to its activity or function and still result in a variant with an acceptable level of equivalent or similar biological activity or function. In some examples, the amino acid residue changes are conservative amino acid substitution, which refers to the amino acid residue of a similar chemical structure to another amino acid residue and the polypeptide function, activity or other biological effect on the properties smaller or substantially no effect. Typically, relatively more substitutions can be made in FR regions, in contrast to CDR regions, as long as they do not adversely impact the binding function and bioactivities of the antibody (such as reducing the binding affinity by more than 50% as compared to the original antibody). In some embodiments, the sequence identity can be about 80%, 82%, 84%, 85%, 86%, 88%, 90%, 92%, 94%, 95%, 96%, 98%, or 99%, or higher, between the reference antibody and the variant. Variants can be prepared according to methods for altering polypeptide sequence known to one of ordinary skills in the art such as those found in references which compile such methods, e.g. Molecular Cloning: A Laboratory Manual, J. Sambrook, et al., eds., Second Edition, Cold Spring Harbor Laboratory Press, Cold Spring Harbor, New York, 1989. For example, conservative substitutions of amino acids include substitutions made amongst amino acids within the following groups: (i) A, G; (ii) S, T; (iii) Q, N; (iv) E, D; (v) M, I, L, V; (vi) F, Y, W; and (vii) K, R, H.

The antibodies described herein may be animal antibodies (e.g., mouse-derived antibodies), chimeric antibodies (e.g., mouse-human chimeric antibodies), humanized antibodies, or human antibodies. The antibodies described herein may also include their antigen-binding fragments e.g. a Fab fragment, a F(ab′)2 fragment, a Fv fragment, a single chain Fv (scFv) and a (scFv)₂. The antibodies or their antigen-binding fragments can be prepared by methods known in the art.

### III. Preparation of Antibodies

Numerous methods conventional in this art are available for obtaining antibodies or antigen-binding fragments thereof.

In some embodiments, the antibodies provided herein may be made by the conventional hybridoma technology. In general, a target antigen e.g. a tumor antigen optionally coupled to a carrier protein, e.g. keyhole limpet hemocyanin (KLH), and/or mixed with an adjuvant, e.g complete Freund's adjuvant, may be used to immunize a host animal for generating antibodies binding to that antigen. Lymphocytes secreting monoclonal antibodies are harvested and fused with myeloma cells to produce hybridoma. Hybridoma clones formed in this manner are then screened to identify and select those that secrete the desired monoclonal antibodies.

In some embodiments, the antibodies provided herein may be prepared via recombinant technology. In related aspects, isolated nucleic acids that encode the disclosed amino acid sequences, together with vectors comprising such nucleic acids and host cells transformed or transfected with the nucleic acids, are also provided.

For examples, nucleic acids comprising nucleotide sequences encoding the heavy and light chain variable regions of such an antibody can be cloned into expression vectors (e.g., a bacterial vector such as an E. coli vector, a yeast vector, a viral vector, or a mammalian vector) via routine technology, and any of the vectors can be introduced into suitable cells (e.g., bacterial cells, yeast cells, plant cells, or mammalian cells) for expression of the antibodies. Examples of nucleotide sequences encoding the heavy and light chain variable regions of the antibodies as described herein are as shown in Table 1 below. Examples of mammalian host cell lines are human embryonic kidney line (293 cells), baby hamster kidney cells (BHK cells), Chinese hamster ovary cells (CHO cells), African green monkey kidney cells (VERO cells), and human liver cells (Hep G2 cells). The recombinant vectors for expression the antibodies described herein typically contain a nucleic acid encoding the antibody amino acid sequences operably linked to a promoter, either constitutive or inducible. Typical vectors contain transcription and translation terminators, initiation sequences, and promoters useful for regulation of the expression of the nucleic acid encoding the antibody. The vectors optionally contain selection markers for both prokaryotic and eukaryotic systems. In some examples, both the heavy and light chain coding sequences are included in the same expression vector. In other examples, each of the heavy and light chains of the antibody is cloned into an individual vector and produced separately, which can be then incubated under suitable conditions for antibody assembly.

The recombinant vectors for expression the antibodies described herein typically contain a nucleic acid encoding the antibody amino acid sequences operably linked to a promoter, either constitutive or inducible. The recombinant antibodies can be produced in prokaryotic or eukaryotic expression systems, such as bacteria, yeast, insect and mammalian cells. Typical vectors contain transcription and translation terminators, initiation sequences, and promoters useful for regulation of the expression of the nucleic acid encoding the antibody. The vectors optionally contain selection markers for both prokaryotic and eukaryotic systems. The antibody protein as produced can be further isolated or purified to obtain preparations that substantially homogeneous for further assays and applications. Suitable purification procedures, for example, may include fractionation on immunoaffinity or ion-exchange columns, ethanol precipitation, sodium dodecyl sulfate polyacrylamide gel electrophoresis (SDS-PAGE), high-performance liquid chromatography (HPLC), ammonium sulfate precipitation, and gel filtration.

When a full-length antibody is desired, coding sequences of any of the V_{H} and V_{L} chains described herein can be linked to the coding sequences of the Fc region of an immunoglobulin and the resultant gene encoding a full-length antibody heavy and light chains can be expressed and assembled in a suitable host cell, e.g., a plant cell, a mammalian cell, a yeast cell, or an insect cell.

Antigen-binding fragments can be prepared via routine methods. For example, F(ab′)₂ fragments can be generated by pepsin digestion of an full-length antibody molecule, and Fab fragments that can be made by reducing the disulfide bridges of F(ab′)₂ fragments. Alternatively, such fragments can also be prepared via recombinant technology by expressing the heavy and light chain fragments in suitable host cells and have them assembled to form the desired antigen-binding fragments either in vivo or in vitro. A single-chain antibody can be prepared via recombinant technology by linking a nucleotide sequence coding for a heavy chain variable region and a nucleotide sequence coding for a light chain variable region. Preferably, a flexible linker is incorporated between the two variable regions.

One antibody can be further modified to conjugate one or more additional elements at the N- and/or C-terminus of the antibody such as another protein and/or a drug or carrier. Preferably, an antibody conjugated with an additional element retains the desired binding specificity and therapeutic effect while providing additional properties resulted from the additional element that aids, for example, in solubility, storage or other handling properties, cell permeability, half-life, reduction in hypersensitivity, controls delivery and/or distribution. Other embodiments include the conjugation of a label e.g. a dye or fluorophore for assays, detection, tracking and the like. In some embodiments, an antibody can be conjugated to an additional element such as a peptide, dye, fluorophore, carbohydrates, anti-cancer agent, lipid, etc. In addition, an antibody can be attached to the surface of a liposome directly via an Fc region, for example, to form immunoliposomes.

### IV. Compositions

According to the present invention, the anti-ENO1 antibody may be formulated with a pharmaceutically acceptable carrier into a composition for purpose of delivery and absorption.

As used herein, “pharmaceutically acceptable” means that the carrier is compatible with an active ingredient in the composition, and preferably can stabilize said active ingredient and is safe to the receiving individual. Said carrier may be a diluent, vehicle, excipient, or matrix to the active ingredient. Typically, a composition comprising an anti-ENO1 antibody as described herein as an active ingredient can be in a form of a solution such as an aqueous solution e.g. a saline solution or it can be provided in powder form. Appropriate excipients also include lactose, sucrose, dextrose, sorbose, mannose, starch, Arabic gum, calcium phosphate, alginates, tragacanth gum, gelatin, calcium silicate, microcrystalline cellulose, polyvinyl pyrrolidone, cellulose, sterilized water, syrup, and methylcellulose. The composition may further contain pharmaceutically acceptable auxiliary substances as required to approximate physiological conditions, for example, pH adjusting and buffering agents, such as sodium acetate, sodium chloride, potassium chloride, calcium chloride, sodium lactate and the like. The form of the composition may be tablets, pills, powder, lozenges, packets, troches, elixers, suspensions, lotions, solutions, syrups, soft and hard gelatin capsules, suppositories, sterilized injection fluid, and packaged powder. The composition of the present invention may be delivered via any physiologically acceptable route, such as oral, parenteral (such as intramuscular, intravenous, subcutaneous, and intraperitoneal), transdermal, suppository, and intranasal methods. In certain embodiments, the composition of the present invention is administered as a liquid injectable formulation which can be provided as a ready-to-use dosage form or as a reconstitutable stable powder.

### V. Treatment

According to the present invention, the anti-ENO1 antibody as described herein can specifically bind to ENO1 and is capable of inhibiting ENO1-downstream signaling pathways, including attenuating HGF-triggered phosphorylation of HGFR and its downstream signaling components, decreasing the association between ENO1 and HGFR, and/or reducing SLUG steady-state protein levels, for example. The anti-ENO1 antibody as described herein thus benefits treatment of diseases and disorders associated with activation of ENO1-downstream signaling.

A method is therefore disclosed herein for inhibiting ENO1-downstream signaling and/or treating a disease or disorder associated with activation of ENO1-downstream signaling. The method involves administering an effective amount of an anti-ENO1 antibody as described herein as an active ingredient or a composition comprising the same to a patient in need thereof. In some embodiments, activation of ENO1-downstream signaling is associated with a proliferation disease or disorder e.g. a cancer and a metastasis thereof. In certain embodiments, the cancer is selected from the group consisting of lung cancer, brain cancer, breast cancer, cervical cancer, colon cancer, gastric cancer, head and neck cancer, kidney cancer, leukemia, liver cancer, ovarian cancer, pancreatic cancer, prostate cancer, skin cancer and testicular cancer which have been observed to have the features of overexpression of ENO1 mRNA or protein. In one particular example, the cancer is lung cancer.

The term “effective amount” used herein refers to the amount of an active ingredient to confer a desired biological effect in a treated subject or cell. The effective amount may change depending on various reasons, such as administration route and frequency, body weight and species of the individual receiving said pharmaceutical, and purpose of administration. Persons skilled in the art may determine the dosage in each case based on the disclosure herein, established methods, and their own experience.

A subject to be treated by the method of treatment as described herein can be a mammal, more preferably a human. Mammals include, but are not limited to, farm animals, sport animals, pets, primates, horses, dogs, cats, mice and rats. A human subject who needs the treatment may be a human patient having, at risk for, or suspected of having a target disease/disorder, such as cancer. A subject suspected of having any of such target disease/disorder might show one or more symptoms of the disease/disorder. A subject at risk for the disease/disorder can be a subject having one or more of the risk factors for that disease/disorder.

In some embodiments, a subject in need to be treated by the method of the present invention possesses cells or tissues that express (e.g. overexpress) ENO1. It is possible to initiate the antibody therapy as described herein prior to the first sign of possible disease or prior to or after diagnosis of a disease.

In some embodiment, the antibody therapy as described herein can be used in combination with one or more cancer therapies known in this art. Such known cancer therapies include surgery, radiation, chemotherapeutic treatment, cell therapy and any combination thereof.

### VI. Diagnostic and Prognostic

ENO1 has been observed in many different tumor types and is known to be highly correlated with reduced survival and poor prognosis. The present invention therefore provides a method of detecting ENO1 using an anti-ENO1 antibody as described herein for both diagnostic and prognostic purposes. In general, the method of detection as described herein involves (i) contacting anti-ENO1 antibody or antigen-binding fragment thereof as described herein with a cell of a subject in need; and (ii) detecting binding of the antibody or antigen-binding fragment thereof to the cell. The cell can be present in vitro where the cell is in a biological sample isolated from a subject in need or in vivo in a subject in need. The subject can be a subject at risk for, or suspected of having a cancer, or a subject afflicted with cancer who may be undergoing cancer treatment. The method of detection as described herein can be useful to identify a subject amendable to cancer therapy and/or to monitor cancer progression or response to therapy.

In some embodiments, when the method of detection is performed in vitro, the biological sample can be any type of samples such as a body fluid sample or a tissue sample in which a cancer cell may be present. Examples of such sample include but not limited to, blood, urine, saliva, cerebrospinal fluid, ascites, lymph fluid, nipple aspirate fluid, bronchoalveolar lavage fluid, semen, or a tissue or biopsy sample from lung, breast, cervix, colon, stomach, liver, ovary, pancreas, prostate, skin and testis. For example, a biological sample is obtained from a subject and the biological sample is analyzed by an immunoassay using an anti-ENO1 antibody as described herein to detect the presence or level of ENO1 in the sample. Examples of immunoassay's include, but are not limited to, Western blot, enzyme-linked immunosorbent assay (ELISA), radioimmunoassay (RIA), radioimmunoprecipitation assay (RIPA), immunofluorescence assay (IFA), ELFA (enzyme-linked fluorescent immunoassay), electrochemiluminescence (ECL), and Capillary gel electrophoresis (CGE).

In some embodiments, when the method of detection is performed in vivo, suitable in vivo imaging technology can be used in whole, live subject in need. For example, an anti-ENO1 antibody as described herein can be detectably labeled and administered to a subject in need and the bound detectably labeled antibody is detected using imaging methods available in the art.

In some embodiments, the method of detection can be used for prognosis purposes. Specifically, the method may further comprise comparing the results of the detection with a reference level of ENO1 and determining prognosis for the subject based on the results of the comparison, wherein an elevated level of ENO1 is indicative of a negative prognosis. In some embodiments, a reference level can refer to the level measured in normal individuals or samples such as tissues or cells that are not diseased (adjacent non-cancerous/normal tissues). In some embodiments, a reference level can be a standard value which may represent the average or median amount of a marker (e.g. ENO1) in a population of cancer patients. Typically, such population of cancer patients are chosen to be matched to the candidate individual in, for example, age, cancer types and/or ethnic background. Preferably, such population of cancer patients and the candidate individual are of the same species (e.g. human).

In some embodiments, the method of detection is performed for a subject afflicted with cancer for the purpose to monitor the cancer progression. Specifically, a biological sample is obtained from a cancer patient from different time points and cancer progression in the patient is determined based on the expression level of ENO1, wherein an elevated level of ENO1 over time indicates cancer progression.

In a particular embodiment, the method of detection comprises
(i) providing a first biological sample from a cancer patient at a first time point;
(ii) providing a second biological sample from the patient at a second time point, which is later than the first time point;
(iii) detecting the levels of ENO1 in the first and second biological samples, wherein the detection is performed by an immunoassay with an antibody or antigen-binding fragment thereof as described herein; and
(iv) determining cancer progression in the patient based on the levels of ENO1 in the first and second biological samples, wherein an elevated level of ENO1 in the second biological sample as compared to that in the first biological sample is indicative of cancer progression.

Also provided is a kit for performing the method of the invention. Specifically, the kit comprises an anti-ENO1 antibody or antigen-binding fragment thereof as described herein or a composition comprising the same and instructions (e.g., written, tape, VCR, CD-ROM, etc.) for using the kit to detect the presence or amount of ENO1 in vitro, e.g. in a biological sample from a subject in need or in a subject in need in vivo. The assay format of the kit can be a chip or an ELISA, for example.

### VII. Screening Methods for an Anti-Cancer Candidate Agent

According to the present invention, binding to ENO1 on the surface of a cancer cell and/or downregulation of ENO1 downstream signaling is critical to inhibition of cancer growth and progression. Therefore, the present invention provides a method for screening for a candidate agent for cancer therapy based on the activity of binding to ENO1 on the surface of a cancer cell and/or downregulating ENO1 downstream signaling.

In particular, the method of the present invention comprises (i) contacting an agent with a cancer cell expressing ENO1 on the cell surface of the cancer cell; (ii) detecting binding of the agent with ENO1 on the cell surface of the cancer cell and/or measuring the levels of one or more ENO1-downstream signaling events/components in the cancer cell; and (iii) determining if the agent is a candidate anti-cancer based on the results of the detection and/or measurement.

In some embodiments, the results of the detection showing that the agent binds to ENO1 on the cell surface indicates that the agent has an anti-cancer effect.

In some embodiments, the results of the measurement showing a decreased level of one or more of the ENO1-downstream signaling events/components in the cancer cell in the presence of the agent as compared to that in the absence of the agent indicates that the agent has an anti-cancer effect.

In some embodiments, the one or more ENO1-downstream signaling events/components comprises HGF-triggered phosphorylation of HGFR, association between ENO1 and HGFR, and/or SLUG steady-state protein.

In some embodiments, the anti-cancer effect comprises inhibition of tumor growth and/or metastasis.

In some embodiments, the cancer is selected from the group consisting of lung cancer, brain cancer, breast cancer, cervical cancer, colon cancer, gastric cancer, head and neck cancer, kidney cancer, leukemia, liver cancer, ovarian cancer, pancreatic cancer, prostate cancer, skin cancer and testicular cancer.

In one particular embodiment, the cancer is lung cancer.

The agent screening as described herein may be performed using an in vitro cell model. A wide variety of assays may be used for this purpose e.g.

immunoprecipitation and western blotting. The agent may then be further analyzed for functional activity e.g. by cell viability assay and/or cell invasion assay and/or using in vivo animal tumor/metastasis model. The agent for screening may be antibodies or small molecular compounds.

The present invention is further illustrated by the following examples, which are provided for the purpose of demonstration rather than limitation. Those of skill in the art should, in light of the present disclosure, appreciate that many changes can be made in the specific embodiments which are disclosed and still obtain a like or similar result without departing from the spirit and scope of the invention.

### EXAMPLES

ENO1 expression is significantly correlated with reduced survival and poor prognosis in many cancer types, including lung cancer. However, the function of ENO1 in carcinogenesis remains elusive, and there is currently no ENO1-based therapeutic drug approved for clinical use. In this study, we found that high expression of ENO1 is present in metastatic lung cancer cell lines and malignant tumors, and it is associated with poor overall survival of lung cancer patients. Functional in vitro studies revealed that knockdown of ENO1 decreases cancer cell proliferation and invasiveness, whereas these processes are enhanced by ENO1 overexpression. Moreover, ENO1 promotes tumor growth in orthotopic xenograft models and enhances lung tumor metastasis in tail-vein-injection models. These effects are likely mediated by upregulation of mesenchymal markers (N-cadherin and vimentin) and the epithelial-mesenchymal transition regulator, SLUG, along with concurrent downregulation of E-cadherin expression. Mechanistically, ENO1 can interact with hepatocyte growth factor receptor (HGFR) and activate HGFR and Wnt signaling by increasing the phosphorylation of HGFR and the Wnt co-receptor, LRP5/6. Activation of these signaling responses decreases GSK-3β activity via Src-PI3K-AKT signaling and inactivation of the β-catenin destruction complex to ultimately upregulate SLUG and β-catenin. Additionally, we generated a chimeric anti-ENO1 monoclonal antibody (chENO1-22) that can decrease cancer cell proliferation and invasion. chENO1-22 inhibits cancer cell invasion by inhibiting ENO1-mediated GSK3β inactivation to promote SLUG protein ubiquitination and degradation. Moreover, chENO1-22 inhibits lung tumor metastasis and prolongs survival time in tail vein injection animal models. Taken together, our findings illuminate the molecular mechanisms underlying the regulation of ENO1 in lung cancer metastasis and introduce a novel antibody for potential therapeutic use in lung cancer.

### 1. Material and Methods

### 1.1 Cell Culture and Reagents

NL-20, CL1-5, H460 cells were purchased from American Type Culture Collection (ATCC) and were authenticated by ATCC based on DNA profile, cytogenetic analysis and isoenzymology. The cells were cultured in accordance with ATCC protocols and were passaged for fewer than 6 months after resuscitation. NL-20 is an immortalized, nontumorigenic human bronchial epithelial cell line derived from normal bronchus. NHBE cells are normal human bronchial epithelial cells, which were purchased from Lonza. Hop62, CL1-0 and CL141 cells were generous gifts from Dr. Pan-Chyr Yang (National Taiwan University). The Gsk-3 inhibitor, BIO, was purchased from Sigma-Aldrich.

### 1.2 Reverse Transcriptase-PCR and Western Blotting

Reverse transcriptase-PCR and immunoblotting were conducted as described previously³⁷. The following primary antibodies were used in immunoblotting assay: anti-ENO1 (11204-1-AP; Proteintech), anti-E-cadherin (24E10; Cell Signaling Technology.), anti-Vimentin (D21H3; Cell Signaling Technology), anti-N-cadherin (610920; BD Biosciences), anti-SNAIL (C15D3; Cell Signaling Technology), anti-TWIST (GTX127310; GeneTex), anti-SLUG (C19G7; Cell Signaling Technology), anti-GSK-3β (27C10; Cell Signaling Technology), Phospho-GSK-3β (Y216) (612312; BD Biosciences), Phospho-GSK-3β (Ser9) (5B3; Cell Signaling Technology), anti-non-phospho (Active) β-Catenin (D13A1; Cell Signaling Technology), anti-β-Catenin (D10A8; Cell Signaling Technology), Phospho-Src Family (Tyr416) (D49G4; Cell Signaling Technology), Phospho-PI3 Kinase p85 (Tyr458)/p55 (Tyr199) (Cell Signaling Technology), Phospho-Akt (Ser473) (Cell Signaling Technology), anti-FLAG (F3165; Sigma-Aldrich) and anti-Myc (9E10; Santa Cruz Biotechnology) and anti-HGFR (DIC2; Cell Signaling Technology), Horseradish peroxidase (HRP)-conjugated rabbit anti-mouse or goat anti-rabbit secondary antibodies (Santa Cruz Biotechnology) were used as appropriate.

### 1.3 Flow Cytometry

Flow cytometry was carried out on a Beckman Dickinson LSR-II flow cytometer (Cornell Biotechnology; Flow Cytometery Core Facility) equipped with 407, 488, 633 nm lasers. Typical single color analysis was conducted. Curve fitting and data analysis were performed using GraphPad Prism (v 6.0). P-values were obtained by unpaired Student's t test. Cell counting was performed with a Countess II (Invitrogen).

### 1.4 Tissue Microarrays and Immunohistochemistry

Tissue microarrays were obtained from commercial sources (SuperBioChips). A total of 9 normal lung samples, 40 primary lung cancer samples, and 10 metastatic lung cancer cells were tested. The primary antibody used for staining was targeted against anti-ENO1 (Abcam Biotechnology). Signal intensities of IHC were graded as 0 (no expression), 1 (faint expression), 2 (moderate expression) and 3 (intense expression) by an independent investigator blinded to group-identifying information. Areas of positive staining, expressed as a percentage, were also evaluated.

### 1.5 Functional Analysis of the Target Protein

Lentiviruses (pLKO.1) containing the shRNA and pLKO.1 empty vector or pLKO-luciferase controls were generated and used to infect cancer cells following standard procedures. Stable transfectants were established by puromycin selection as described previously³⁸. In addition to overexpression of the gene products after transfection, the target gene effects were evaluated by functional assays. Changes in cellular morphology, proliferation rate and invasion ability were assessed by previously described protocols³⁸.

### 1.6 Cell Viability

Cells were harvested by trypsinization and were resuspended in RPMI containing 10% FBS. The cells were then plated at a density of 3000 per well in 96-well plates and incubated overnight. Four replicates were treated with each concentration of drug; PBS buffer was added to the control group. After adding WST-1, the reagent was kept in the 96-well plate for 1 h. The absorbance at 450 nm was monitored.

### 1.7 Matrigel Transwell Cell Invasion Assay

Matrigel invasion assay was performed using a 24-well transwell plates (Costar) with polycarbonate filters (pore size, 8 μm). Matrigel was attenuated to 1 mg/ml with 4° C. serum-free medium on ice and was added to the upper chamber of the plates. After 30 min incubation in a 37° C. incubator, a cell suspension with 1×10⁵ cells/ml was added. For the lower chamber, 800 μl medium with 10% FBS was added. After 16-24 h the cells were fixed with 95% methanol for 5 min. Crystal violet (0.1%) was used to stain cells. The transwell plate was then observed under a microscope.

### 1.8 In Vivo Orthotopic Lung Tumor and Metastasis Model

Female non-obese diabetic/severe combined immunodeficiency (NOD-SCID) mice were used to establish orthotopic lung tumor and metastatic lung cancer xenograft models. For the orthotopic xenograft model, 1×10⁵ CL1-5 cells stably expressing luciferase and infected with shENO1 or control shCtrl were orthotopically injected into the lung parenchyma of NOD-SCID mice. For the metastasis model, 1×10⁶ CL1-5 cells stably expressing luciferase and infected with shENO1 or control shCtrl were injected into mice by tail vein injection. All animal studies were approved by the Institutional Animal Care and Use Committee of the National Defense Medical Center, Taipei, Taiwan.

### 1.9 Immunofluorescence Analysis

Cells were grown to the appropriate level of confluence and treated as indicated. At the conclusion of the experiment, cells were fixed by addition of paraformaldehyde (PFA) at 4° C. for 20 min. PFA was removed, after which cells were washed three times with PBS and blocked with 2 ml imaging blocking buffer (3% BSA in PBS) at 37° C. for 1 h. Plates were washed two times with PBS, then treated with primary antibody at room temperature for 1 h. Primary antibody was removed, and plates were washed two times with PBS. Then secondary antibody was added for 1 h. Plates were washed one time in PBS containing 1 mg/ml DAPI, then once with PBS. Samples were stored at 4° C. until imaging.

### 1.10 TOP/FOP Wnt Activity Assay

CL1-5 cells (2×10⁴ cells per well) were seeded in 24-well plates. Plasmids were transfected into cells using Lipofectamine 3000 (Invitrogen). A luciferase reporter (TOPflash or FOPflash) or was co-transfected with a pGL4.74 hRluc-TK construct (Renilla luciferase). The luciferase activity was measured by a dual-luciferase reporter assay system (Promega, Madison, WI, USA). Transcriptional activity was determined as the expression of firefly luciferase normalized to the Renilla luciferase levels at 48 h post-transfection. Measurements were made with a dual luciferase reporter assay kit (Promega).

### 1.11 Phospho-RTK Array Analysis

The Human Phospho-RTK Array Kit (R&D Systems) was used to determine the relative levels of tyrosine phosphorylation for 49 distinct RTKs, according to the manufacturer's protocol. Briefly, the arrays were incubated with 300 μg of protein lysate overnight at 4° C. after blocking for 1 h with Array Buffer 1. The arrays were washed and incubated with an anti-Phospho-Tyrosine-HRP Detection Antibody. Signals were generated with a Chemi Reagent Mix and captured using a chemiluminescence imaging system (GE ImageQuant LAS4000). The intensity of each phospho-RTK array signal was quantitated with ImageJ software; relative intensities of the average signal from each pair of duplicate spots were determined in relation to the negative control spots.

### 1.12 GST Pull-Down

In the GST pull-down assay, His-HGFR was incubated with glutathione bead-bound GST-ENO1 or bead-bound GST alone (control) in pull-down buffer (Pierce™ GST Protein Interaction Pull-Down kit) at 4° C. for 1 h.

### 1.13 Immunoprecipitation (IP)-Western Blotting

Protein lysate was incubated with anti-FLAG agarose beads (A2220; Sigma-Aldrich, USA), anti-Myc agarose beads (C3956; Sigma-Aldrich, USA) for co-IP experiments, or anti-SLUG (C19G7; Cell Signaling Technology, Inc. USA) for ubiquitination (Ub) assays. The following primary antibodies were used in immunoblotting assay: anti-ENO1 (11204-1-AP; Proteintech), anti-SLUG (C19G7; Cell Signaling Technology), anti-FLAG (F3165; Sigma-Aldrich), anti-Myc (9E10; Santa Cruz Biotechnology), anti-Ubiquitin (P4D1; Cell Signaling Technology) and anti-HGFR (DIC2; Cell Signaling Technology). Horseradish peroxidase (HRP)-conjugated rabbit anti-mouse or goat anti-rabbit secondary antibodies (Santa Cruz Biotechnology, USA) were used as appropriate.

### 1.14 Generation of Monoclonal Antibodies Against Tumor Antigen

Generation of mAbs against tumor antigens was performed according to standard procedures³⁹, with some modifications⁴⁰⁻⁴². Briefly, the spleen of the immunized mouse was removed, and splenocytes were fused with NSI/1-Ab4-1 (NS-1) myeloma cells. The splenocytes and the myeloma cells were washed twice with serum-free DMEM medium. The final pellet was mixed in a 15-ml conical tube, and 1 ml 50% (v/v) polyethylene glycol (GIBCO BRL) was added over 1 min with gentle stirring. The mixture was diluted through slow (1 min) addition of 1 ml of DMEM twice, followed by slow addition (2 min) of 8 ml of DMEM medium without serum. The mixture was centrifuged, and the fused cell pellet was resuspended in DMEM medium supplemented with 15% FBS, hypoxanthine-aminopterin-thymidine (HAT) medium, and hybridoma cloning factor and distributed in the 96-well tissue culture plates. Hybridoma colonies were screened by ELISA for secretion of mAbs that bound to tumor antigens. Selected clones were subcloned by limiting dilution. Final hybridoma clones were isotyped using an isotyping kit. Ascitic fluids were produced in pristane-primed BALB/c mice. The hybridoma cell lines were grown in DMEM medium with 10% heat-inactivated FBS. The monoclonal antibodies were affinity purified with protein G sepharose 4B gels. ELISA assays and Western blots were used to measure the activity and specificity of the antibodies.

### 1.15 ELISA

Cell culture (96-well) plates (Corning Costar) were fixed with 2% paraformaldehyde and blocked with 1% bovine serum albumin (BSA). Cells were added to the plates and incubated for 1 h. The plates were subsequently washed with PBS containing 0.1% (w/v) Tween-20 (PBST0.1), followed by incubation with horseradish peroxidase-conjugated anti-mouse IgG (115-035-062; Jackson ImmunoResearch Laboratories) for 1 h. After washing, the plates were incubated with substrate solution (o-phenylenediamine dihydrochloride; P6787, Sigma). The reaction was stopped by the addition of 3 N HCl, and signals were detected using a microplate reader at 490 nm.

### 1.16 Immunogold Labeling

CL1-5 cells were fixed with 4% formaldehyde and processed for cryosectioning. Sections were blocked with BSA, followed by a labeling for ENO1 with the chENO1 antibody-22 (40 μg/ml) or NHIgG. After post-fixation the samples were stained and examined with a transmission electron microscope.

### 1.17 Statistical Analysis

Data are presented as mean±SEM. Comparisons of data between two groups were made with Student's t-test. Statistical analyses were performed with GraphPad Prism software, version 5.0. All statistical tests were two-sided, and P-values<0.05 were considered statistically significant.

### 2. Results

### 2.1 ENO1 is Highly Expressed in Metastatic Cancer Cell Lines and Patients, and its High Expression is Associated With Worse Overall Survival of Lung Cancer Patients

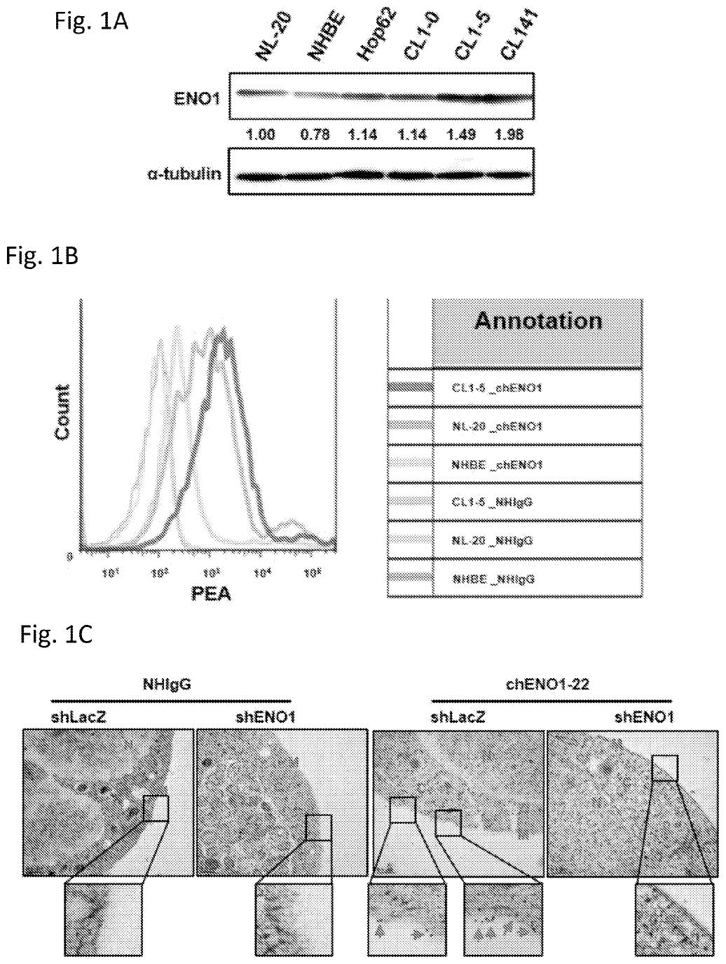

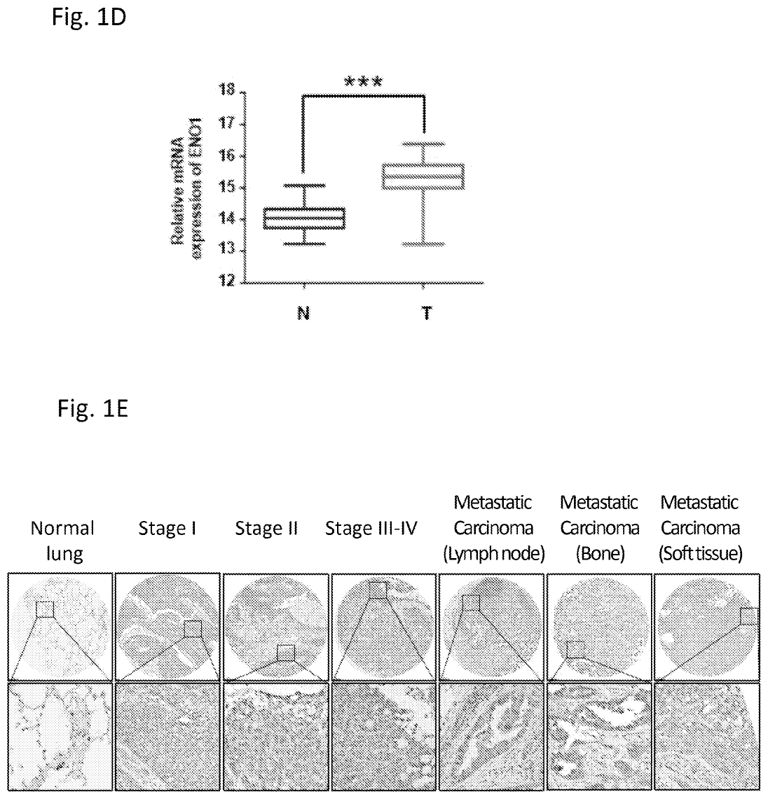

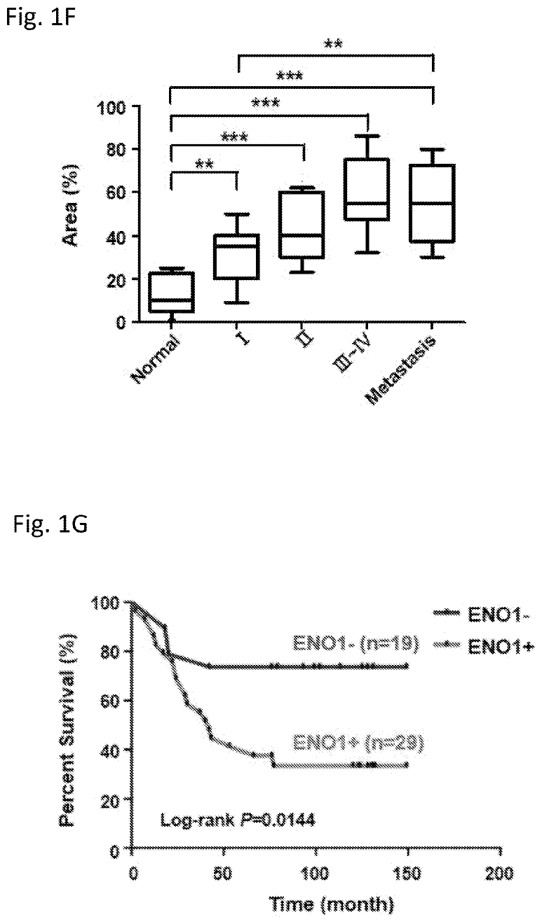

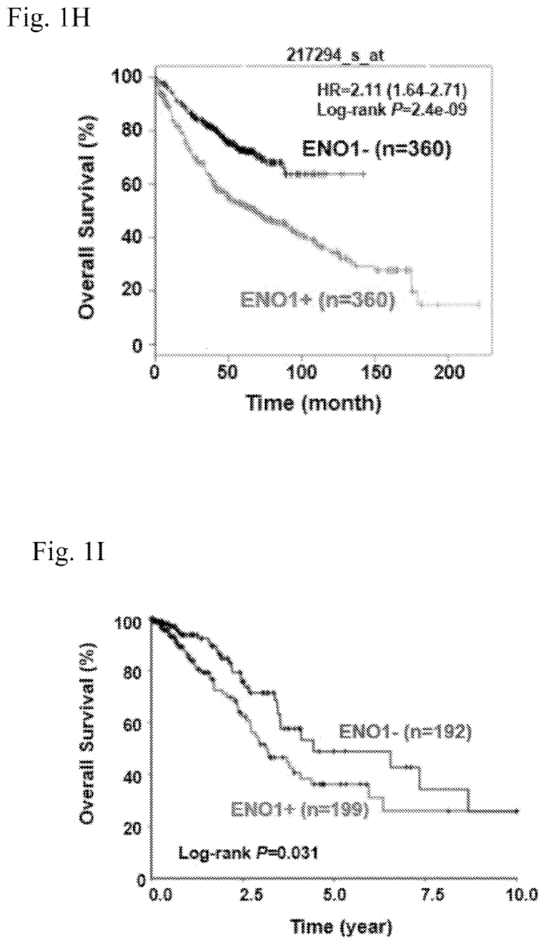

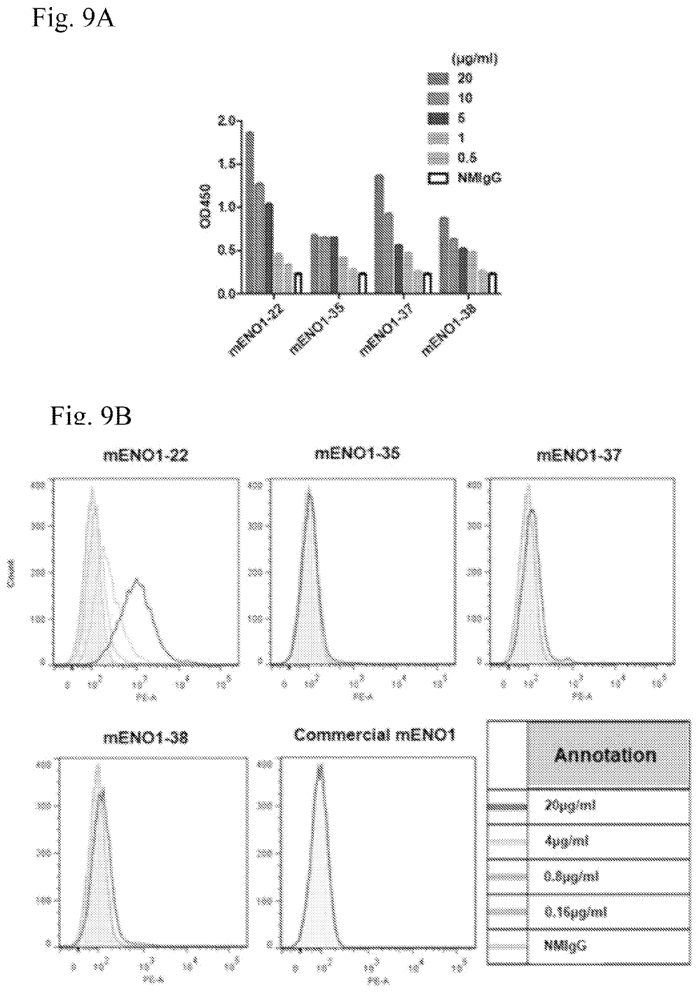

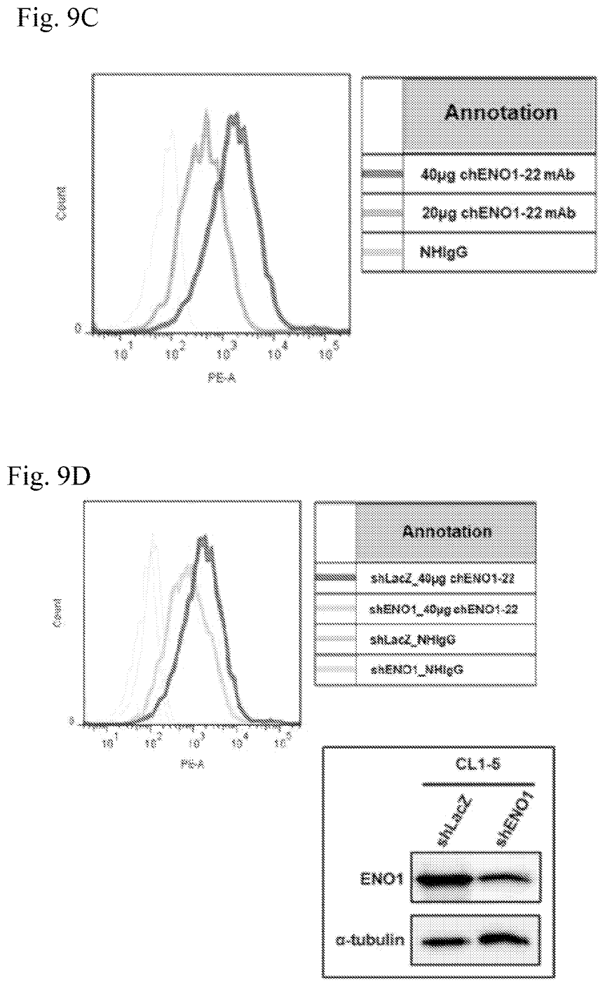

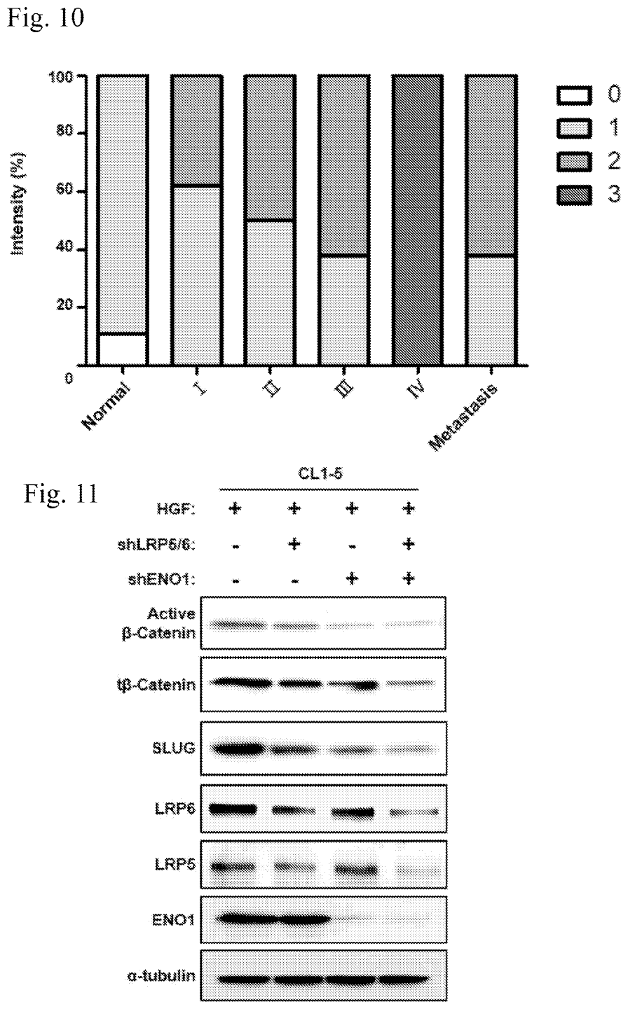

To assess the correlation between ENO1 expression and lung cancer malignancy, we measured ENO1 protein levels in a panel of lung cancer cells and clinical NSCLC specimens. ENO1 protein level was elevated in highly metastatic lung cancer cell lines (CL1-5 and CL141) compared to non-metastatic cell lines (CL1-0) and Hop62), and only weak expression was detected in normal human bronchial epithelial (NHBE) cells or NL-20 immortalized human bronchial epithelial cells (FIG. 1A). In addition, the surface expression of ENO1 was upregulated in CL1-5 cells compared with NHBE or NL-20 cells, according to flow cytometry analysis (FIG. 1B) with a chimeric ENO1 antibody (FIG. 9). Moreover, we performed immunogold labelling assays to confirm ENO1 protein localization. Expectedly, ENO1 was detected on the cell surface, in the cytoplasm and in the nucleus of CL1-5 cells (FIG. 1C). In clinical tumor samples from NSCLC patients, increased ENO1 expression was associated with increased TNM and metastatic stage, but ENO1 mRNA and protein were not abundant in normal lung tissue (FIGS. 1D-1F and FIG. 10). Furthermore, survival analysis based on the protein expression level of ENO1 revealed the significant decrease in the survival rate of patients with NSCLC that showed high ENO1 expression as compared with those with low ENO1 expression (FIG. 1G). Moreover, high mRNA expression of ENO1 was strongly associated with worse overall survival for NSCLC patients according to the Kaplan-Meier plotter database (KM plotter, http://kmplot.com/analysis/) (FIG. 1H) and TCGA dataset (FIG. 1I). Taken together, these findings indicate a strong correlation between high expression of ENO1 and lung cancer malignancy.

### 2.2 ENO1 Enhances Lung Cancer Cell Viability and Invasion

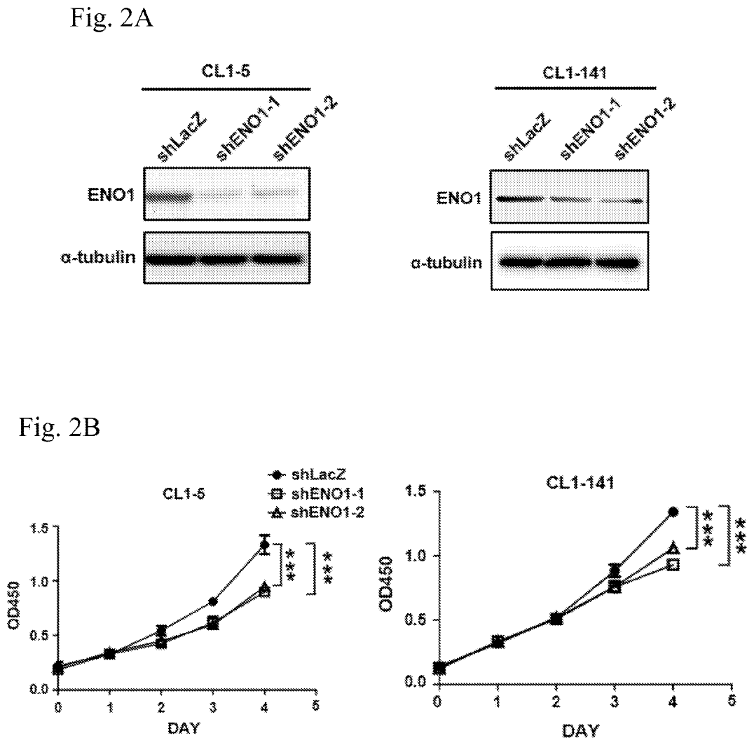

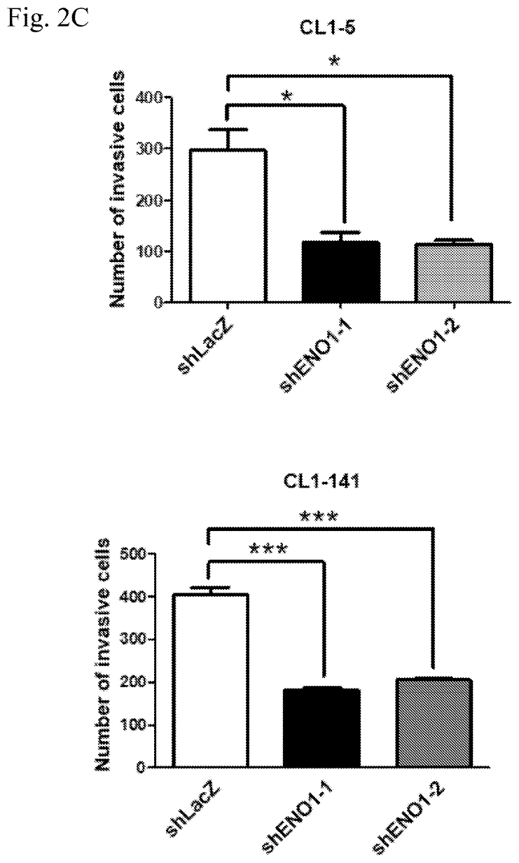

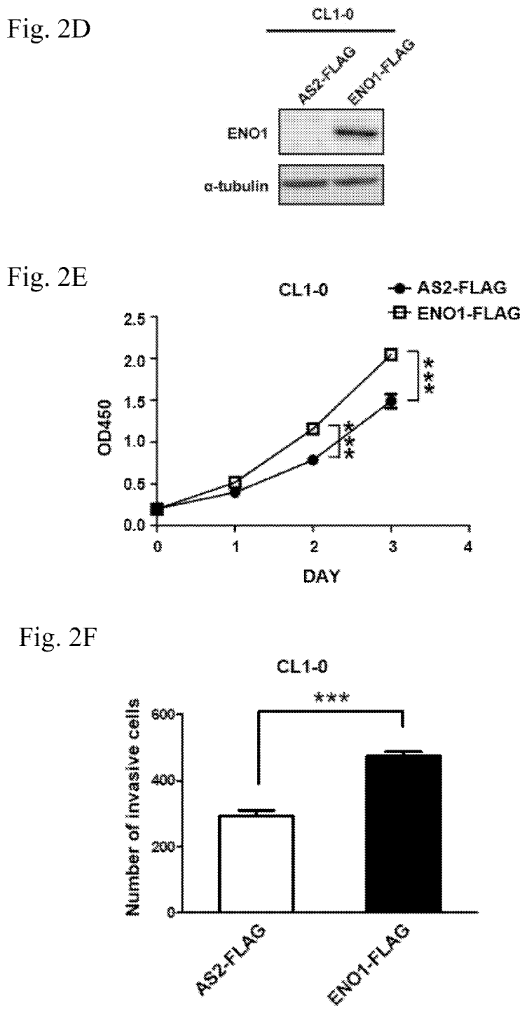

To investigate the biological function of ENO1 in lung cancer cells in vitro, we established a constitutive ENO1-knockdown system. CL1-5 or CL-141 cells were infected with lentivirus harboring either one of two small hairpin RNAs (shRNAs) that target distinct coding sequences of ENO1 (shENO1-1, shENO1-2) or control non-targeting shRNA (shLacZ) (FIG. 2A). Knockdown of ENO1 significantly decreased cell viability (FIG. 2B) and cell invasion (FIG. 2C). We also stably overexpressed ENO1 in CL1-0) cells, which normally show low ENO1 expression (FIG. 2D), and we found that ENO1 overexpression significantly enhanced cell viability (FIG. 2E) while increasing the number of invading cells (FIG. 2F). Collectively, these results suggest that the ENO1 increases the viability and invasion of lung cancer cells in vitro.

### 2.3 ENO1 Promotes Lung Cancer Tumorigenicity and Metastasis in Xenograft Models

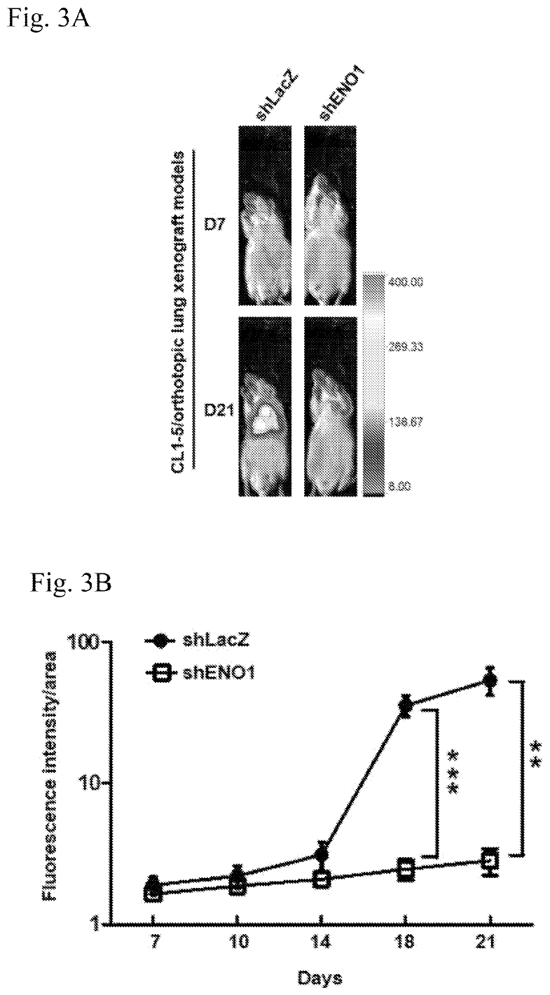

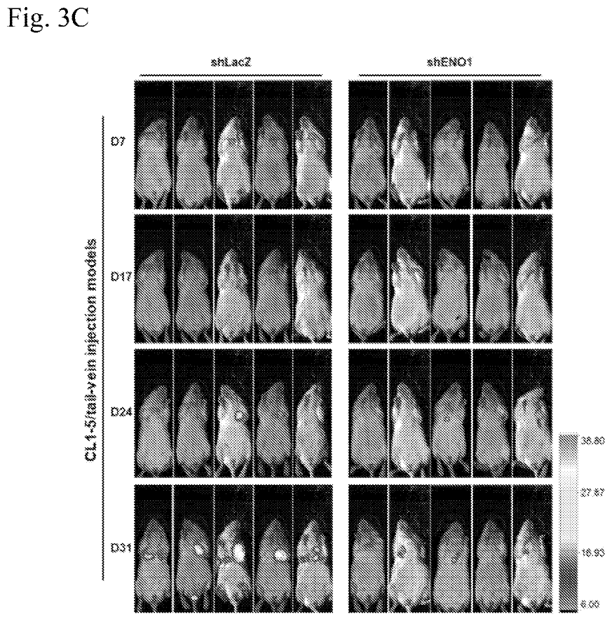

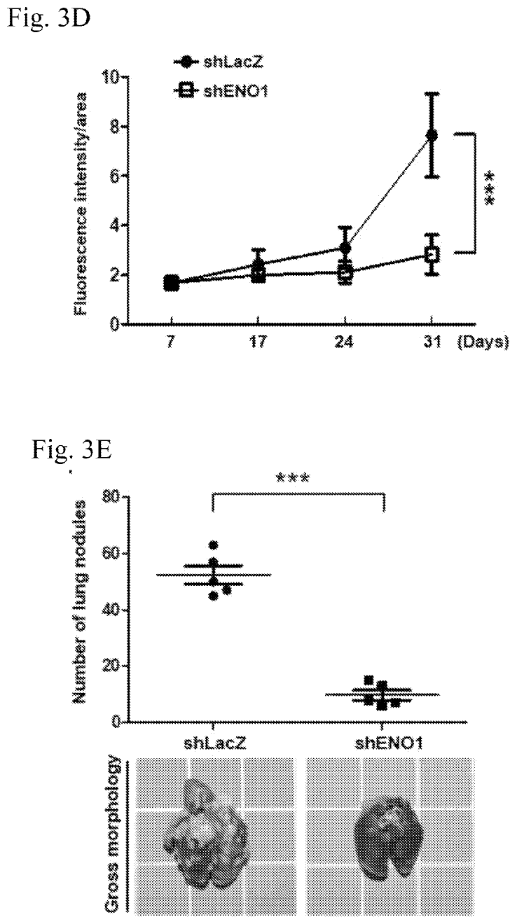

Tumor growth, invasion and metastasis are key steps in tumor progression. First to explore the effect of ENO1 on tumorigenicity in vivo, we established an orthotopic xenograft model. One hundred thousand CL1-5 cells stably expressing luciferase and infected with either shENO1 or shCtrl were orthotopically injected into the lung parenchyma of NOD-SCID mice. Using luciferin-based bioluminescence to detect tumor size, we found that mice injected with ENO1-knockdown cells had lower luciferase signal than those injected with shCtrl-infected cells, indicting a lower tumor growth rate for ENO1-knockdown tumors (FIGS. 3A and 3B). Lung tissues were collected 21 days after implantation, and hematoxylin and eosin staining was used to detect lung nodules.

To further test whether ENO1 may affect lung cancer metastasis, we investigated lung nodule formation in NOD-SCID mice intravenously injected with CL1-5 cells stably expressing luciferase and infected with shENO1 or shCtrl. Mouse lungs were collected 31 days after injection, and lung nodules were counted. As shown in FIGS. 3C and 3D, there were stronger luciferase signals in lungs of mice injected with control shCtrl cells compared to the ENO1-knockdown group. In addition, ENO1-knockdown cells created fewer metastatic lung nodules than control cells (FIG. 3E). Taken together, these data demonstrate that ENO1 plays a critical role in promoting tumor formation and metastatic behavior of lung cancer cells in xenograft models.

### 2.4 ENO1 Enhances Cancer Cell Invasion via HGFR- and WNT-Driven Epithelial-Mesenchymal Transition

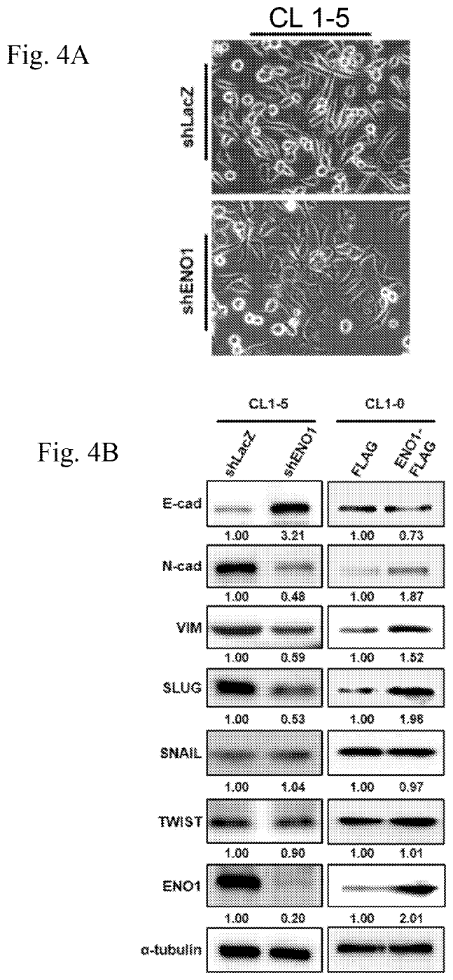

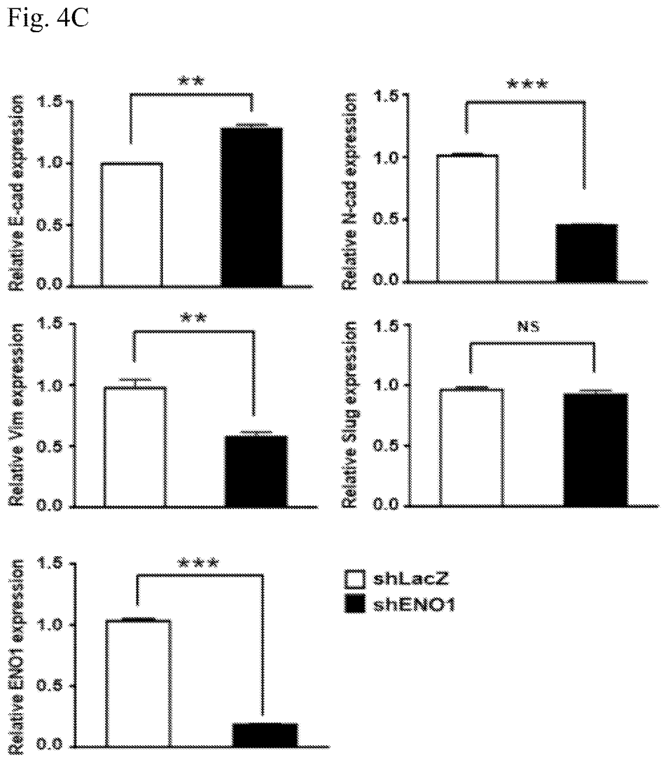

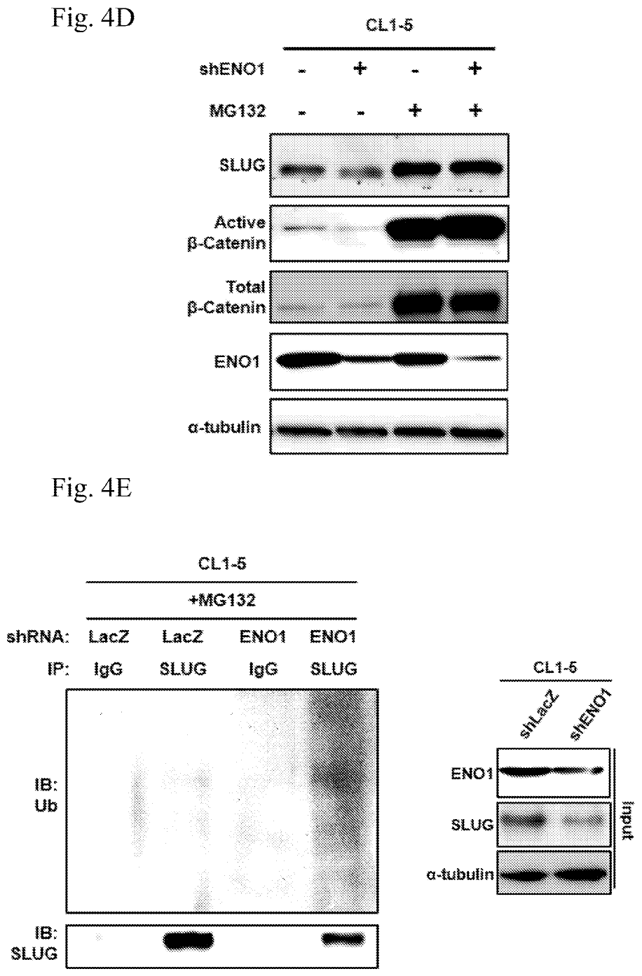

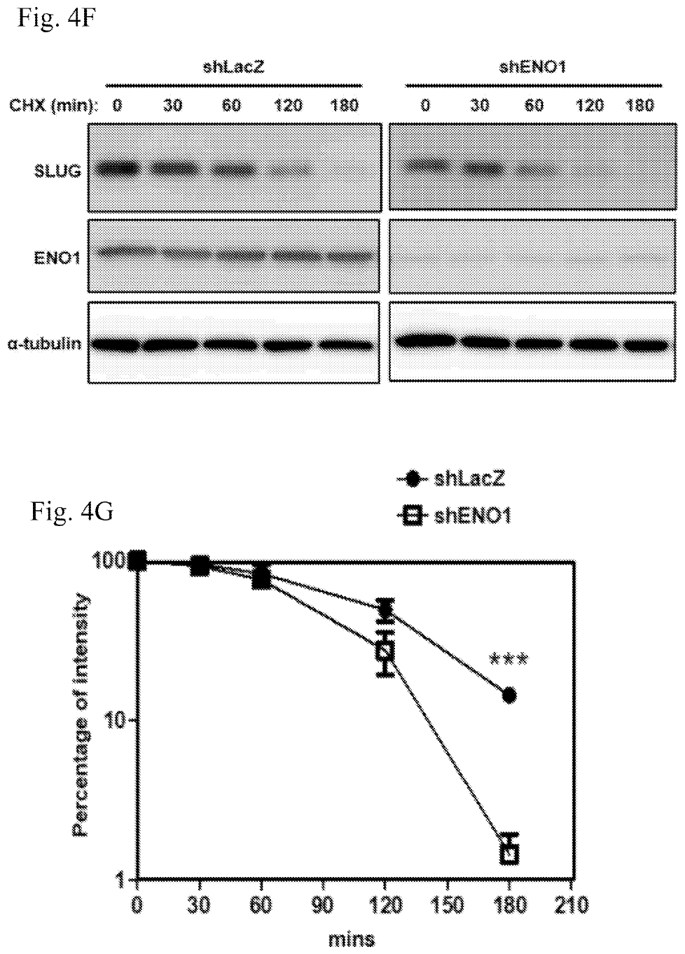

Previous studies have shown that EMT is associated with cancer invasion, metastasis and progression. Because ENO1-knockdown CL1-5 cells exhibited a more epithelial-like phenotype compared with control cells, which displayed fibroblast-like mesenchymal features (FIG. 4A), we decided to profile the expression EMT-related genes in ENO1-knockdown and control cells. We found that ENO1 knockdown inhibited the expression of the mesenchymal markers, N-cadherin, vimentin, as well as the protein level of the EMT regulator, SLUG, while enhancing E-cadherin expression (FIG. 4B). ENO1-mediated upregulation of SLUG expression was also observed in CL1-5 cells by immunofluorescence. In contrast, ectopic expression of ENO1 in CL1-0) cells increased the levels of N-cadherin, vimentin and SLUG, while decreasing E-cadherin. Interestingly, ENO1 did not affect SLUG mRNA level (FIG. 4C). Treatment with MG132 (inhibitor of the 26S proteasome) increased SLUG steady-state protein levels, indicating that the protein level is largely controlled by degradation (FIG. 4D). Knockdown of ENO1 led to increased levels of ubiquitylated SLUG compared with control cells (FIG. 4E), and it shortened the SLUG protein half-life, according to the protein levels after cyclohexamide treatment (FIGS. 4F and 4G). Based on these results, we concluded that ENO1 regulates SLUG protein stability but not its expression.

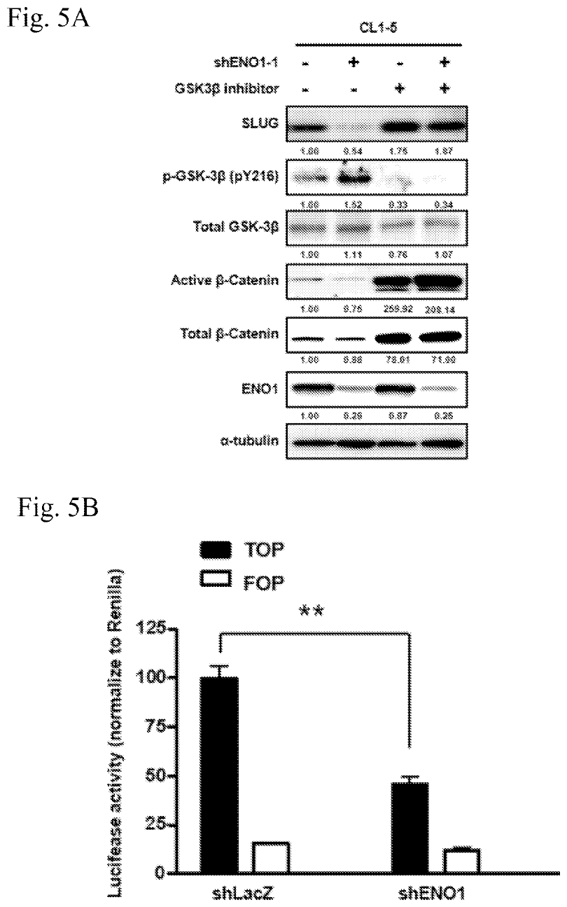

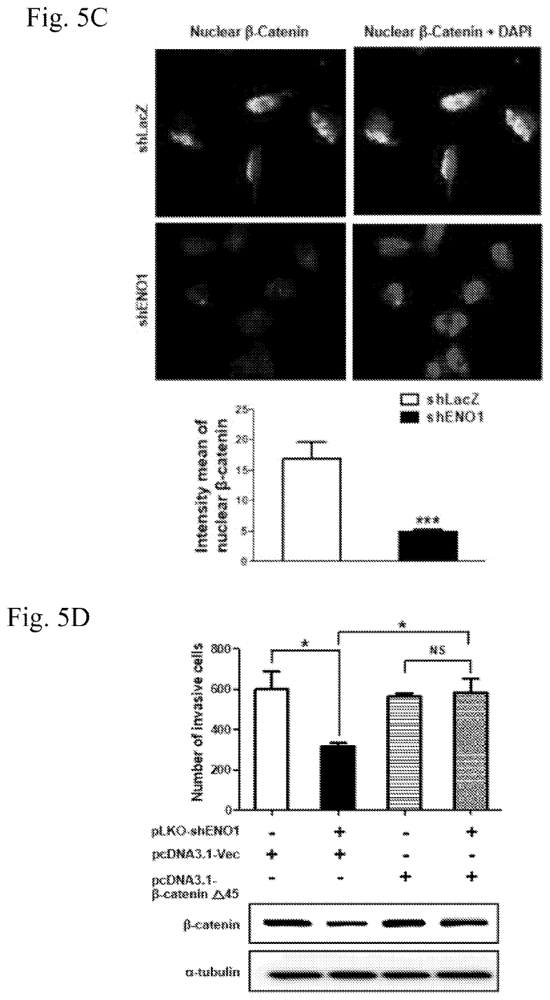

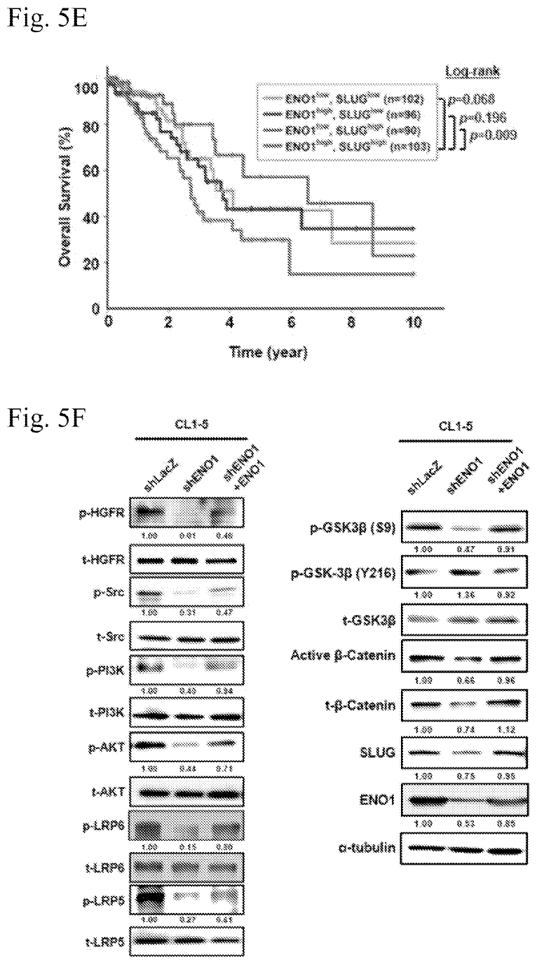

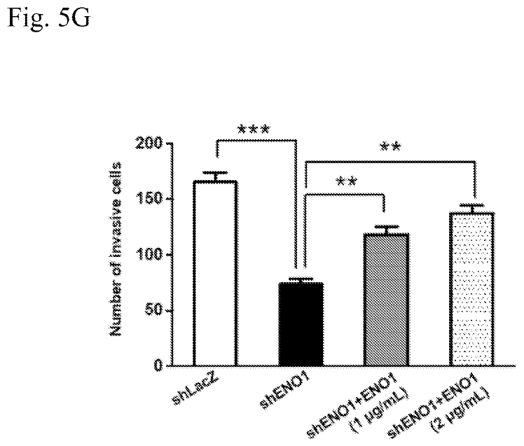

Since the Wnt signaling antagonist GSK-3β is known to affect SLUG protein turnover via phosphorylation and ubiquitin-mediated proteolysis, we hypothesized that ENO1 stabilized SLUG by regulating GSK-3β. Indeed, SLUG was upregulated in ENO1-knockdown cells after treatment with a GSK3β inhibitor; meanwhile, the active form of β-catenin was also increased (FIG. 5A). Interestingly, we found that knockdown of ENO1 enhanced GSK-3β (FIG. 5A), and it also decreased Wnt signaling activity, as measured by the TOP/FOP luciferase reporter assays (FIG. 5B). Confocal immunofluorescence revealed that ENO1 knockdown also reduced nuclear β-catenin levels (FIG. 5C). To further test whether β-catenin and SLUG participate in ENO1-induced cell invasion, we overexpressed a constitutively active β-catenin (β-cateninΔ45) or vector control and found that overexpression of β-cateninΔ45 restored invasion capacity of ENO1 knockdown cells, but did not affect invasion of control cells (FIG. 5D). In addition, we also overexpressed mutant SLUG (Slug-4SA; SLUG protein with four GSK-3β-targeted serine residues replaced by alanines) or wild-type Slug (Slug-WT)⁴³ and found that overexpression of mutant Slug restored invasion capacity and cell proliferation in ENO1 knockdown cells, but wild-type Slug did not. Next, we analyzed SLUG protein levels in a panel of lung cancer cells, finding that SLUG protein level was elevated in highly metastatic lung cancer cell lines (CL1-5 and CL141) compared to non-metastatic cell lines (CL1-0) and Hop62) and normal human bronchial epithelial (NHBE) cells. The expression panel was reminiscent of ENO1 expression in cell lines (FIG. 1A). Patient samples were stratified in low and high ENO1 and SLUG groups, and survival of each group was analyzed. The analysis showed that patients with high ENO1 and high SLUG expression had the worst overall survival (FIG. 5E). Together, these data support the idea that ENO1 promotes malignant cell invasion via the GSK3β-SLUG signaling axis.

Because the phenotypes of ENO1-expressing cells are often caused by receptor tyrosine kinase (RTK) activation, we further examined whether any RTKs might be involved in ENO1 regulation of the GSK3β-SLUG axis. Human Phospho-RTK arrays were treated with cell lysates from shLacZ- and shENO1-infected CL1-5 cells, revealing an extreme difference in the level of HGFR phosphorylation between the groups. This result was confirmed by Western blotting (knockdown of ENO1 decreased the level of phosphorylated HGFR), which was also used to probe the known downstream signals. HGFR phosphorylation was coincident with activation of the Src-PI3K-AKT pathway and inactivation of the GSK-3β destruction complex, as well as stabilized SLUG and β-catenin in CL1-5 and CL141 cells (FIG. 5F). Furthermore, purified recombinant ENO1 could rescue shENO1 knockdown cell phenotypes, such as HGFR and WNT downstream signaling (FIG. 5F), and also rescue shENO1 knockdown-suppressive cell invasive ability (FIG. 5G). HGF is known to be an important transactivator of canonical Wnt signaling, an effect that is mediated by HGFR-stimulated GSK-3β-dependent LRP5/6 phosphorylation⁴⁴, and our data showed that ENO1 could enhance Wnt signaling (FIGS. 5B-D and 5F). Therefore, we tested whether ENO1 activates Wnt signaling via LRP5/6 phosphorylation. Indeed, knockdown of ENO1 decreased the phosphorylation of LRP5/6, but it did not affect the LRP5/6 protein expression (FIG. 5F).

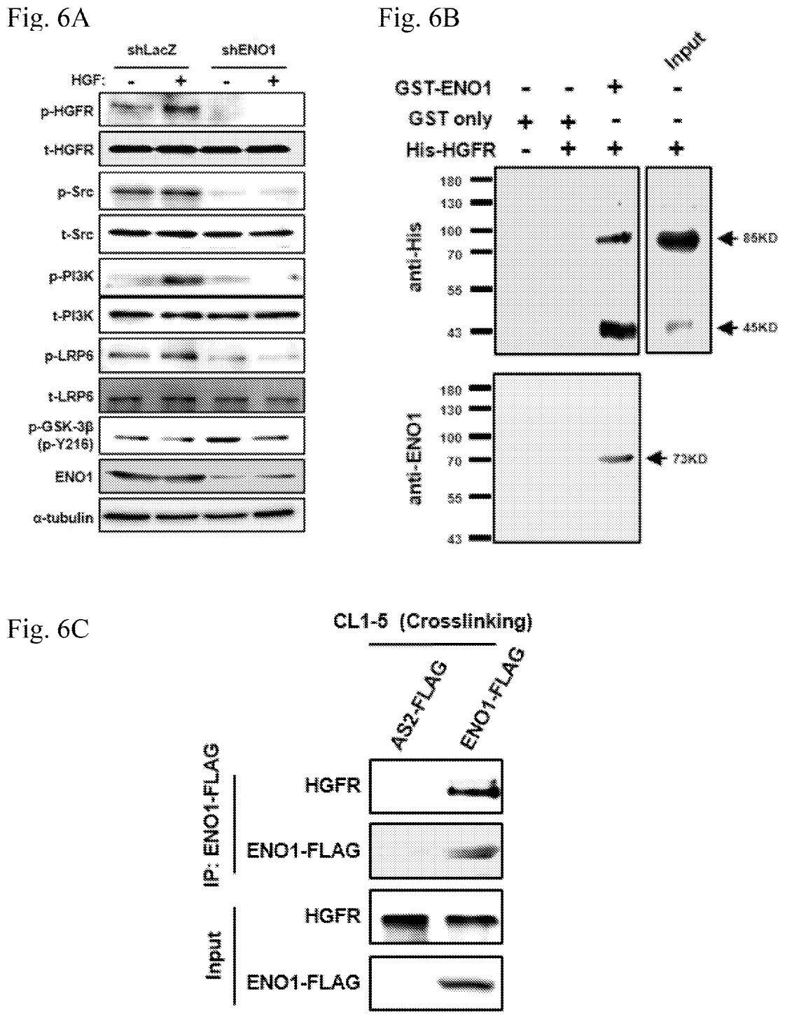

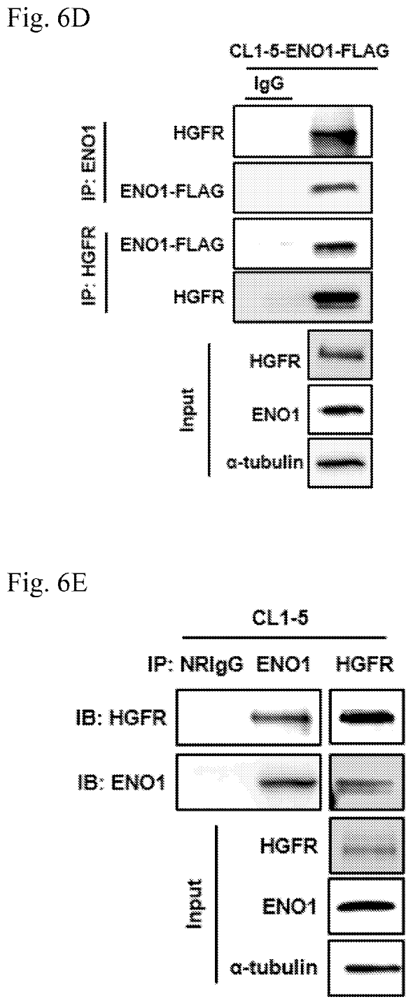

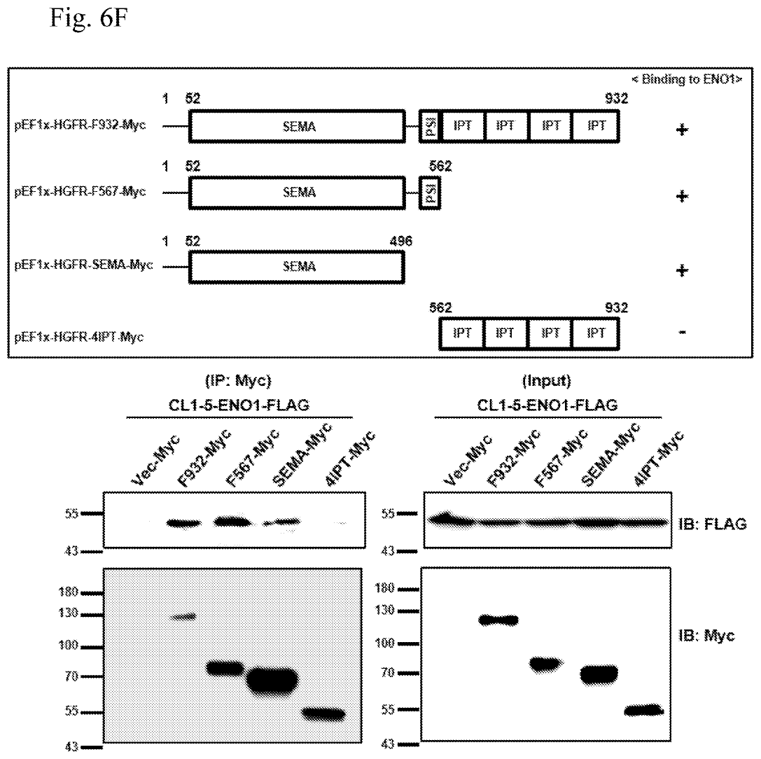

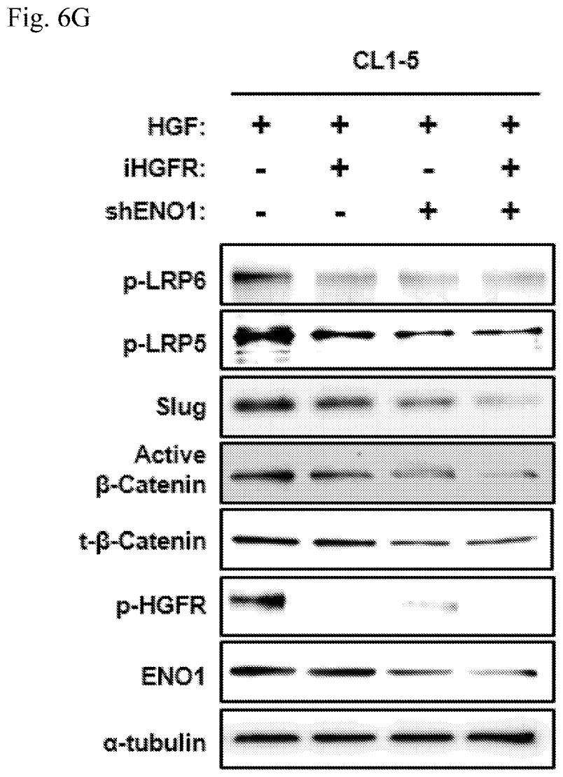

Surprisingly, we also found that ENO1 is essential for HGFR activation since knockdown of ENO1 prevented HGF activation of HGFR and its downstream signaling (FIG. 6A). Thus, we sought to define the mechanism through which ENO1 induces HGFR phosphorylation. We performed GST pull-down assays, in which GST-ENO1 fusion protein or GST alone were incubated with purified His-tagged HGFR, to check for a direct interaction between purified ENO1 and HGFR protein. The pull-down results showed that under our experimental conditions, His-HGFR bound to GST-ENO1 but not GST alone (FIG. 6B). Then we used non-cell permeable crosslinking agents, DTSSP, to further confirm ENO1 bound to HGFR on the cell surface by membrane protein extraction and IP-Western blotting (FIG. 6C). Furthermore, we performed co-immunoprecipitation (Co-IP) of exogenous (FIG. 6D) and endogenous ENO1 with HGFR to validate the interaction between ENO1 and HGFR (FIG. 6E). To further verify the interactions, we also generated several Myc-tagged HGFR truncated mutants, as illustrated in the upper scheme of FIG. 6F. Co-IP assays showed that SEMA domain corresponding to amino acids 52-496 of HGFR was required for the interaction with ENO1 (FIG. 6F). The interaction between ENO1 and HGFR was evident. Therefore, we propose ENO1 may activate HGFR by a direct protein-protein interaction, and the ENO1-induced phosphorylation of LRP5/6 may be dependent on HGFR activation. We also found that ENO1-induced HGFR activation is essential for LRP5/6-mediated stabilization of SLUG and β-catenin; inhibition of HGFR activity in ENO1-expressing cells decreased LRP5/6 phosphorylation (FIG. 6G), and knockdown of both LRP5 and LRP6 decreased ENO1-induced SLUG and β-catenin stabilization (FIG. 11). In total, these data show that ENO1 enhances cancer cell invasion by regulating HGFR- and WNT-driven EMT.

### 2.5 Generation and Characterization of a Therapeutic Monoclonal Antibody Against ENO1

Although many reports have shown that expression of ENO1 significantly correlates with reduced survival and poor prognosis in many cancer types, such as lung cancer¹², there is currently no therapeutic antibody against ENO1 approved for clinical use. Hence, we generated anti-ENO1 mAbs that may be useful for cancer research and therapy. We identified four candidate anti-ENO1 mAbs through mouse hybridoma screening. Our results show that all four anti-ENO1 mAbs possess high binding activity to CL1-5 cells by cellular ELISA assay (FIG. 9A). Importantly, flow cytometry experiments indicated that anti-ENO1 mAb-22 (ENO1-22) can reliably detect ENO1 on the surface of CL1-5 cells (FIG. 9B). We identified the amino acid sequences in the V_{H} and V_{L} domains of the anti-ENO1 mAb-22 antibody from hybridoma cells (Table 1).

TABLE 1
V_{H} domain

| FW1 | CDR1 | FW2 | CDR2 |
|---|---|---|---|
| QVQLQQSGPEVVRPG | DYAMH | WVKQSHA | VISTYNG |
| VSVKISCKGSGYTFT | (SEQ ID | KSLEWIG | NTNYNQK |
| (SEQ ID NO: 1) | NO: 2) | (SEQ ID | FKG |
| | | NO: 3) | (SEQ ID |
| | | | NO: 4) |
| FW3 | CDR3 | FW4 | |
| KATMTVDKSSSTAYM | SPLRY | WGQGTTL | |
| ELARLTSEDSAIYYC | (SEQ ID | TVSS | |
| AR | NO: 6) | (SEQ ID | |
| (SEQ ID NO: 5) | | NO: 7) | |

V_{L} domain

| FW1 | CDR1 | FW2 | CDR2 |
|---|---|---|---|
| QIVLSQSPAILSASP | RASSSVS | WYQQKPG | ATSNLAS |
| GEKVTMTC | YMH | SSPKPWI | (SEQ ID |
| (SEQ ID NO: 8) | (SEQ ID | Y | NO: 11) |
| | NO: 9) | (SEQ ID | |
| | | NO: 10) | |
| FW3 | CDR3 | FW4 | |
| GVPARFSGSGSGTSY | QQWSSNP | FGAGTKL | |
| SLTISRVEAEDAATY | LT | ELK | |
| YC | (SEQ ID | (SEQ ID | |
| (SEQ ID NO: 12) | NO: 13) | NO: 14) | |

Full-length amino acid sequences
of heavy chain and light chain

| | |
|---|---|
| heavy chain | QVQLQQSGPEVVRPGVSVKISCKGSGYTFTDYA |
| | MHWVKQSHAKSLEWIGVISTYNGNTNYNQKFKG |
| | KATMTVDKSSSTAYMELARLTSEDSAIYYCARS |
| | PLRYWGQGTTLTVSS (SEQ ID NO: 15) |
| light chain | QIVLSQSPAILSASPGEKVTMTCRASSSVSYMH |
| | WYQQKPGSSPKPWIYATSNLASGVPARFSGSGS |
| | GTSYSLTISRVEAEDAATYYCQQWSSNPLTFGA |
| | GTKLELK (SEQ ID NO: 16) |

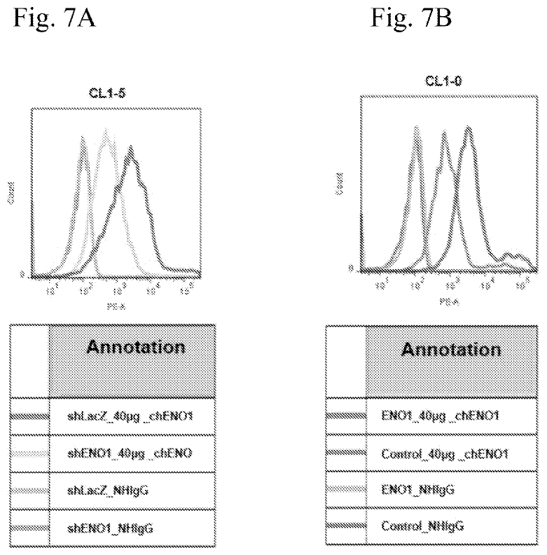

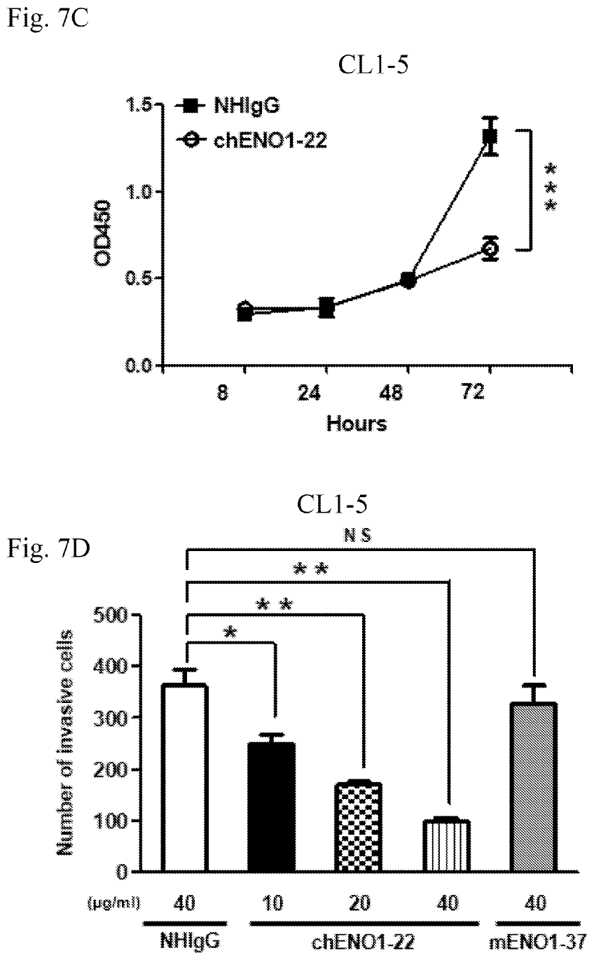

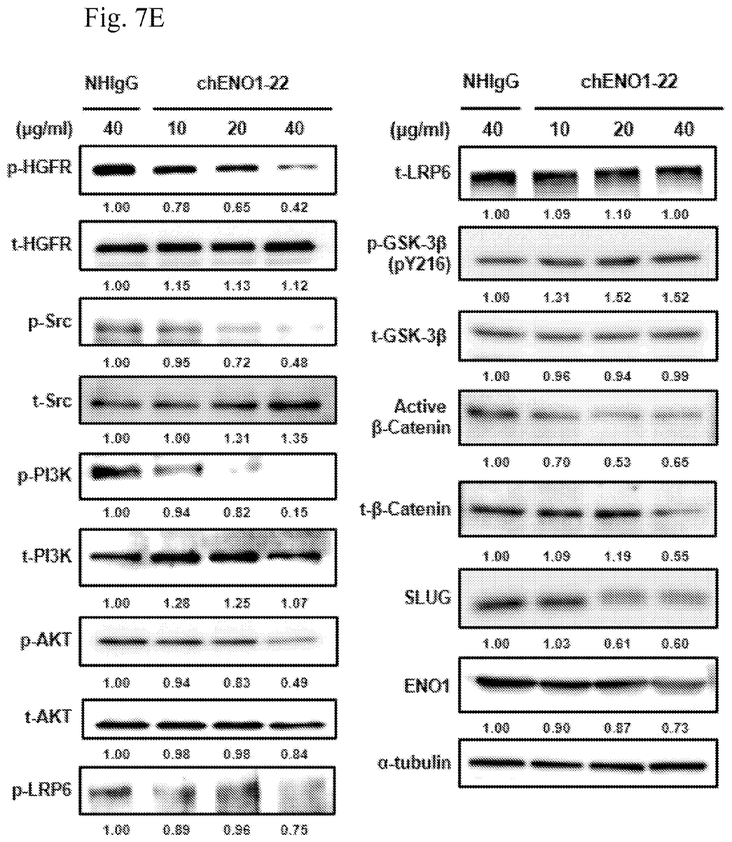

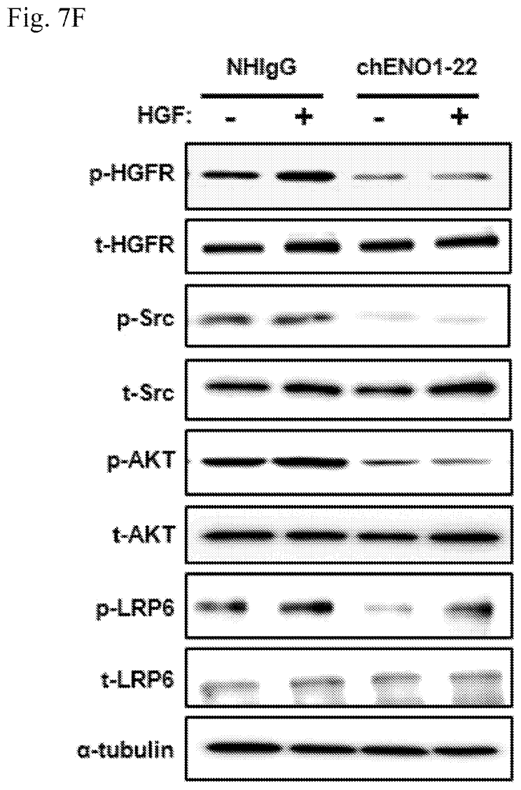

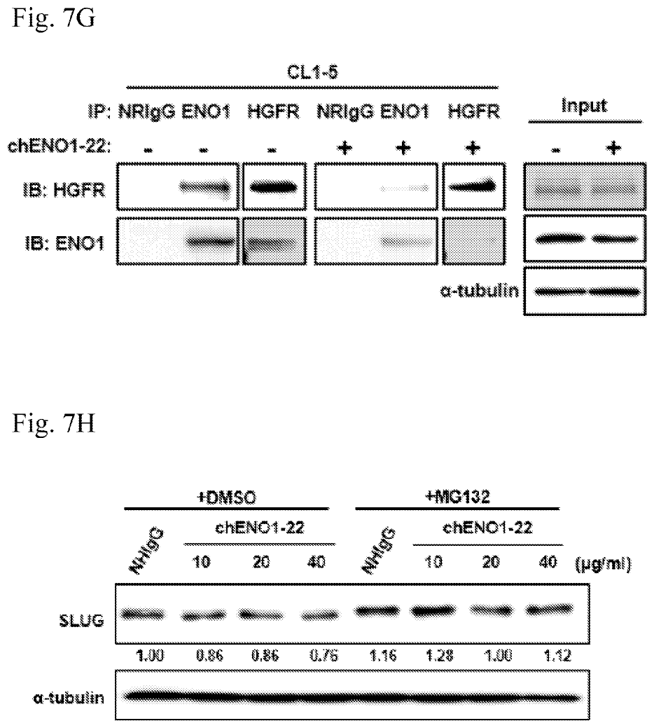

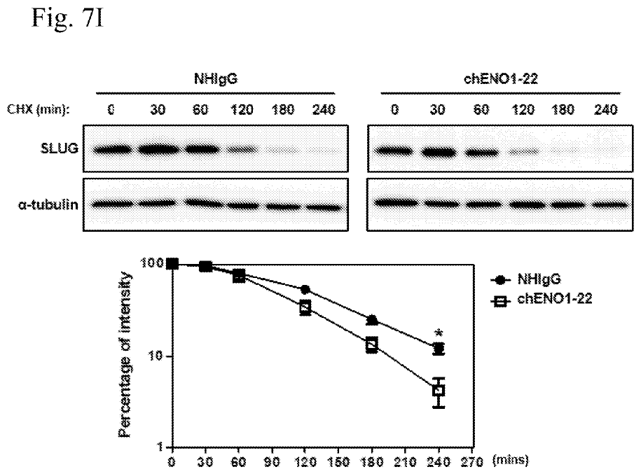

Then we cloned the variable domain of anti-ENO1 mAb-22 antibody into a human IgG Fc vector to generate chimeric ENO1-22 (chENO1-22). We validated the binding activity of chENO1-22, finding that the antibody retained high affinity to live CL1-5 cells (FIG. 9C), and the binding specificity of ENO1 was validated with ENO1-KD cells (FIG. 9D). In addition, we performed immunogold labelling assays to confirm ENO1 protein localization. Expectedly, ENO1 was detected on the cell surface, in the cytoplasm and in the nucleus of CL1-5 cells (FIG. 1C); the binding specificity of ENO1 was then validated with ENO1-knockdown CL1-5 cells (FIG. 7A) or ENO1-overexpressing CL1-0 cells (FIG. 7B).

Next, we evaluated the anti-cancer potential of chENO1-22, including anti-proliferation and anti-invasion effects. chENO1-22 inhibited tumor cell growth (FIG. 7C) and cancer cell invasion in vitro (FIG. 7D). but mENO1-37, which couldn't detect surface form ENO1, couldn't inhibit cancer cell invasion (FIG. 7D). We next sought to test whether chENO1-22 anti-carcinogenic effects are due to its inhibition of ENO1-downstream signaling pathways. Indeed, chENO1-22 treatment inhibited downstream signals of ENO1-HGFR, including the levels of SLUG and active β-catenin (FIG. 7E). Moreover, chENO1-22 treatment attenuated HGF-triggered phosphorylation of HGFR and its downstream signaling components (FIG. 7F). These results were similar to shENO1 knockdown. The mAb also decreased the association between ENO1 and HGFR, as detected by co-IP of endogenous proteins (FIG. 7G). SLUG steady-state protein levels were reduced by treatment with chENO1-22, and treatment with MG132 increased SLUG steady-state protein levels (FIG. 7H). In addition, chENO1-22 shortened the SLUG protein half-life, as assessed with cycloheximide treatment (FIG. 7I). These data show that chENO1-22 inhibition of lung cancer invasion likely occurs as a result of suppressed ENO1-HGFR axis signaling, which allows rapid SLUG protein degradation.

### 2.6 chENO1-22 Antibody Decreases Lung Cancer Cell Metastasis In Vivo

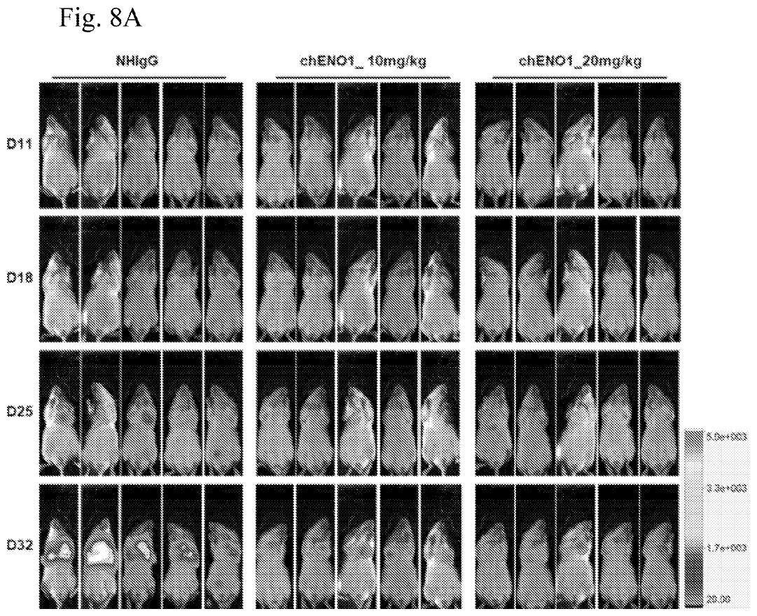

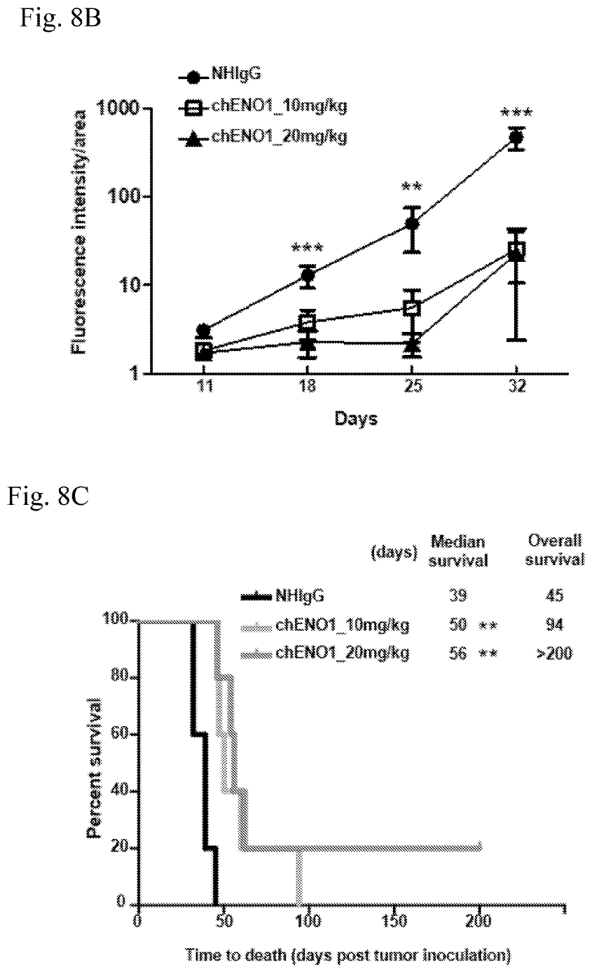

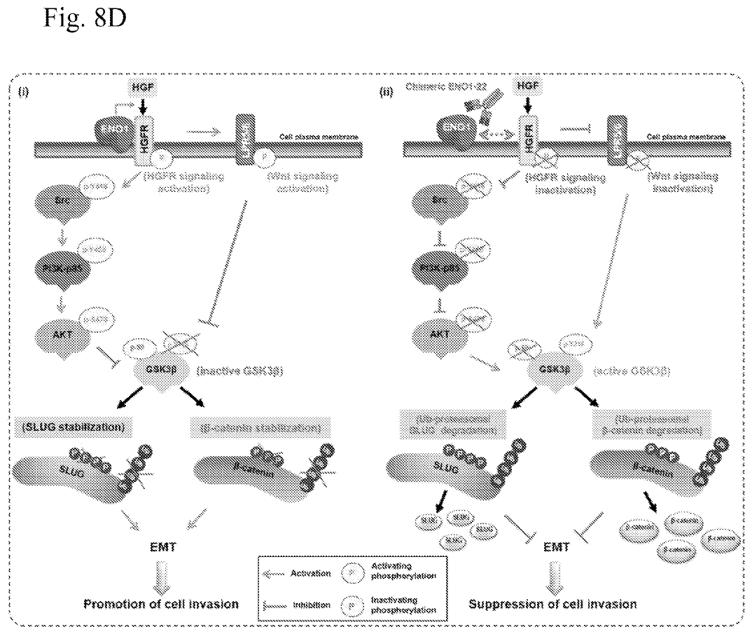

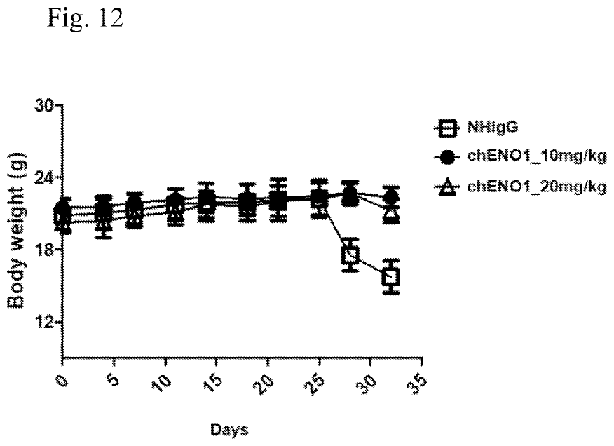

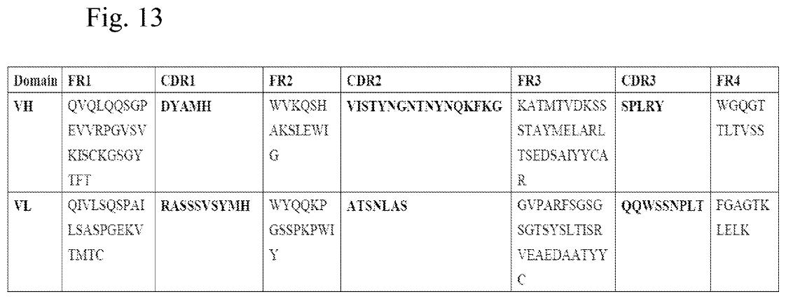

We then wanted to further analyze the therapeutic effects on tumor metastasis in vivo, so we injected CL1-5 luciferase cells into NOD-SCID mice via the tail vein to establish a metastatic xenograft model: the injected mice were subsequently treated with control normal human IgG (NHIgG) or chENO1-22 (10 or 20 mg/kg). After 4 weeks of mAb treatment, weaker luciferase signals were seen in the lungs of mice receiving chENO1-22 compared to NHIgG, no matter whether the dose was 10 or 20 mg/kg (FIGS. 8A and 8B). Thus, chENO1-22 treatment significantly inhibited lung tumor metastasis. In addition, chENO1-22-treated mice maintained a normal body weight (FIG. 12) and showed improved survival compared to NHIgG-treated mice (FIG. 8C).

Based on these findings, we present a proposed model of how ENO1 promotes cell invasion through HGFR-Src-PI3K-AKT-GSK3β-SLUG and WNT-GSK3β-SLUG signaling axes in FIG. 8D.

### 3. Discussion

Our results showed that the ENO1 protein was highly expressed in NSCLC cells but weakly expressed in normal lung tissue. Additionally, high ENO1 expression was positively associated with TMN stage and poor outcomes of patients with lung cancer. These findings suggest that ENO1 is a potentially valuable diagnostic biomarker and/or therapeutic target for lung cancer. The invasion-metastasis cascade can be simplified as two key steps: (i) cancer cell dissemination from the primary tumor to distant tissues and (ii) colonization of distant organs by invading cancer cells, which form micrometastases²². Expression of ENO1-targeting shRNA suppressed lung cancer cell invasion capacity in vitro and disrupted the formation of micrometastases and the metastatic colonization of tumor cells in vivo. Thus, ENO1 may play an important role in lung cancer metastasis. Moreover, ENO1-targeting shRNA suppressed lung cancer growth in an orthotopic tumor model, suggesting that ENO1 may also participate in lung cancer progression. Together, these findings confirm that ENO1 may serve as a therapeutic target for lung cancer.

In this study, we uncovered a few novel features of how ENO1 participates in carcinogenesis. Although several studies previously showed that ENO1 can modulate intravascular and pericellular fibrinolytic activity to facilitate cell migration and cancer metastasis⁶⁻⁸, the detailed signaling mechanism was unclear. In this study we show that a direct protein-protein interaction between ENO1 and HGFR stimulates HGFR signaling. In addition, HGFR was previously known to induce GSK3-dependent LRP5/6 phosphorylation⁴⁴, and we further found that ENO1 triggers HGFR activity to activate LRP5/6-GSK3β-β-catenin canonical Wnt pathway. Thus, we revealed that ENO1 regulates the complex Wnt-/β-catenin pathway through Wnt co-receptor LRP5/6-GSK3β signaling. Moreover, we discovered the interaction between ENO1 and HGFR triggers an HGFR-Src-PI3K-AKT-GSK3β signaling axis. Intriguingly, these two pathways (Wnt and AKT) coordinately stabilize β-catenin and SLUG to promote EMT.

An altered cadherin profile is a well-established hallmark of EMT. Loss of E-cadherin represents a key step in the acquisition of invasiveness, and an increase in N-cadherin signifies a pro-invasive phenotype: this sort of “cadherin switching” is associated with a poor clinical prognosis in many cancers⁴⁵. In the current study, we show that knockdown of ENO1 suppresses mesenchymal transformation by reducing mesenchymal genes (N-cadherin, Vimentin, and SLUG) and upregulating the epithelial gene, E-cadherin. These results reveal a previously undescribed molecular mechanism by which ENO1 participates in lung cancer progression. Slug, an EMT-TF, has been shown to transcriptionally suppress the expression of E-cadherin²² and promote cancer invasion and metastasis in various types of cancers^{46,47}. Previous studies showed that the SLUG-E-cadherin axis is associated with cancer metastasis and poor clinical outcomes in multiple types of NSCLC^{46,48}, suggesting that SLUG is critically involved in lung cancer progression. Indeed, our study indicates that knockdown of SLUG decreases lung cancer cell invasion, and it is involved in ENO1-promoted cell invasion. Moreover, we found that NSCLC patients in the ENO1+ and SLUG+ group had the worst overall survival (P=0.022; FIG. 5D), demonstrating the vital role of ENO1 and SLUG in NSCLC outcome.

We also demonstrated that ENO1 is localized to the cell membrane of lung cancer cells, which was confirmed by flow cytometry and immunogold assays. This observation suggests ENO1 may be a targetable therapeutic target in lung cancer. Next, we generated a therapeutic mAb against ENO1, chENO1-22, which we used to reduce tumor growth and block tumor metastasis in a lung cancer xenograft model. A potential mechanism of action for chENO1-22 is the inhibition of ENO1-HGFR signaling. Since chENO1-22 reduced tumor progression and metastatic colonization in this study, it suggests that this mAb may be translatable to clinical application.

### REFERENCES

1. Gatenby, R. A. & Gillies, R. J. Why do cancers have high aerobic glycolysis? Nat Rev Cancer 4, 891-899 (2004).
2. Hsiao, K. C., et al. Surface alpha-enolase promotes extracellular matrix degradation and tumor metastasis and represents a new therapeutic target. PLoS One 8, e69354 (2013).
3. Diaz-Ramos, A., Roig-Borrellas, A., Garcia-Melero, A. & Lopez-Alemany, R. alpha-Enolase, a multifunctional protein: its role on pathophysiological situations. J Biomed Biotechnol 2012, 156795 (2012).
4. Capello, M., Ferri-Borgogno, S., Cappello, P. & Novelli, F. alpha-Enolase: a promising therapeutic and diagnostic tumor target. FEBS J 278, 1064-1074 (2011).
5. Saldanha, R. G., et al. Proteomic identification of lynchpin urokinase plasminogen activator receptor protein interactions associated with epithelial cancer malignancy. J Proteome Res 6, 1016-1028 (2007).
6. Fu, Q. F., et al. Alpha-enolase promotes cell glycolysis, growth, migration, and invasion in non-small cell lung cancer through FAK-mediated PI3K/AKT pathway. J Hematol Oncol 8, 22 (2015).
7. Zhan, P., et al. alpha-enolase promotes tumorigenesis and metastasis via regulating AMPK/mTOR pathway in colorectal cancer. Mol Carcinog 56, 1427-1437 (2017).
8. Sun, L., et al. Alpha-enolase promotes gastric cancer cell proliferation and metastasis via regulating AKT signaling pathway. European journal of pharmacology 845, 8-15 (2019).
9. Takashima, M., et al. Overexpression of alpha enolase in hepatitis C virus-related hepatocellular carcinoma: association with tumor progression as determined by proteomic analysis. Proteomics 5, 1686-1692 (2005).
10. Tomaino, B., et al. Circulating autoantibodies to phosphorylated alpha-enolase are a hallmark of pancreatic cancer. J Proteome Res 10, 105-112 (2011).
11. Tsai, S. T., et al. ENO1, a potential prognostic head and neck cancer marker, promotes transformation partly via chemokine CCL20 induction. Eur J Cancer 46, 1712-1723 (2010).
12. Chang, G. C., et al. Identification of alpha-enolase as an autoantigen in lung cancer: its overexpression is associated with clinical outcomes. Clin Cancer Res 12, 5746-5754 (2006).
13. Qian, X., et al. Enolase 1 stimulates glycolysis to promote chemoresistance in gastric cancer. Oncotarget 8, 47691-47708 (2017).
14. Wang, H. & Unternaehrer, J. J. Epithelial-mesenchymal Transition and Cancer Stem Cells: At the Crossroads of Differentiation and Dedifferentiation. Dev Dyn 248, 10-20 (2019).
15. Baulida, J. Epithelial-to-mesenchymal transition transcription factors in cancer-associated fibroblasts. Mol Oncol 11, 847-859 (2017).
16. Yao, L., et al. Paracrine signaling during ZEB1-mediated epithelial-mesenchymal transition augments local myofibroblast differentiation in lung fibrosis. Cell Death Differ 26, 943-957 (2019).
17. Mir, N., Jayachandran, A., Dhungel, B., Shrestha, R. & Steel, J. C. Epithelial-to-Mesenchymal Transition: A Mediator of Sorafenib Resistance in Advanced Hepatocellular Carcinoma. Curr Cancer Drug Targets 17, 698-706 (2017).
18. Lovisa, S., Genovese, G. & Danese, S. Role of Epithelial-to-Mesenchymal Transition in Inflammatory Bowel Disease. J Crohns Colitis 13, 659-668 (2019).
19. Yan, L., Xu, F. & Dai, C. L. Relationship between epithelial-to-mesenchymal transition and the inflammatory microenvironment of hepatocellular carcinoma. J Exp Clin Cancer Res 37, 203 (2018).
20. De Matteis, S., et al. Advances in Molecular Mechanisms and Immunotherapy Involving the Immune Cell-Promoted Epithelial-to-Mesenchymal Transition in Lung Cancer. J Oncol 2019, 7475364 (2019).
21. Nieto, M. A., Huang, R. Y., Jackson, R. A. & Thiery, J. P. Emt: 2016. Cell 166, 21-45 (2016).
22. Brabletz, T., Kalluri, R., Nieto, M. A. & Weinberg, R. A. EMT in cancer. Nat Rev Cancer 18, 128-134 (2018).
23. Nieszporek, A., Skrzypek, K., Adamek, G. & Majka, M. Molecular mechanisms of epithelial to mesenchymal transition in tumor metastasis. Acta Biochim Pol 66, 509-520 (2019).
24. Lambert, A. W., Pattabiraman, D. R. & Weinberg, R. A. Emerging Biological Principles of Metastasis. Cell 168, 670-691 (2017).
25. Zhang, Y. & Weinberg, R. A. Epithelial-to-mesenchymal transition in cancer: complexity and opportunities. Front Med 12, 361-373 (2018).
26. Loh, C. Y., et al. The E-Cadherin and N-Cadherin Switch in Epithelial-to-Mesenchymal Transition: Signaling, Therapeutic Implications, and Challenges. Cells 8(2019).
27. Baarsma, H. A., Konigshoff, M. & Gosens, R. The WNT signaling pathway from ligand secretion to gene transcription: molecular mechanisms and pharmacological targets. Pharmacol Ther 138, 66-83 (2013).
28. Volckaert, T. & De Langhe, S. P. Wnt and FGF mediated epithelial-mesenchymal crosstalk during lung development. Dev Dyn 244, 342-366 (2015).
29 Heuberger, J. & Birchmeier, W. Interplay of cadherin-mediated cell adhesion and canonical Wnt signaling. Cold Spring Harb Perspect Biol 2, a002915 (2010).
30. Chang, Y. W., et al. Diverse Targets of beta-Catenin during the Epithelial-Mesenchymal Transition Define Cancer Stem Cells and Predict Disease Relapse. Cancer research 75, 3398-3410 (2015).
31. Parikh, R. A., Wang, P., Beumer, J. H., Chu, E. & Appleman, L. J. The potential roles of hepatocyte growth factor (HGF)-MET pathway inhibitors in cancer treatment. Onco Targets Ther 7, 969-983 (2014).
32. Uehara, Y., et al. Placental defect and embryonic lethality in mice lacking hepatocyte growth factor/scatter factor. Nature 373, 702-705 (1995).
33. Tsarfaty, I., et al. The Met proto-oncogene mesenchymal to epithelial cell conversion. Science 263, 98-101 (1994).
34. Maroun, C. R. & Rowlands, T. The Met receptor tyrosine kinase: a key player in oncogenesis and drug resistance. Pharmacol Ther 142, 316-338 (2014).
35. Nakashiro, K., et al. Phenotypic switch from paracrine to autocrine role of hepatocyte growth factor in an androgen-independent human prostatic carcinoma cell line, CWR22R. Am J Pathol 165, 533-540 (2004).
36. Wu, C. H., Kuo, Y. H., Hong, R. L. & Wu, H. C. alpha-Enolase-binding peptide enhances drug delivery efficiency and therapeutic efficacy against colorectal cancer. Sci Transl Med 7, 290ra291 (2015).
37. Tsao, C. M., et al. SOX1 functions as a tumor suppressor by antagonizing the WNT/beta-catenin signaling pathway in hepatocellular carcinoma. Hepatology 56, 2277-2287 (2012).
38. Lin, C. W., et al. Epithelial cell adhesion molecule regulates tumor initiation and tumorigenesis via activating reprogramming factors and epithelial-mesenchymal transition gene expression in colon cancer. J Biol Chem 287, 39449-39459 (2012).
39. Kohler, G. & Milstein, C. Continuous cultures of fused cells secreting antibody of predefined specificity. Nature 256, 495-497 (1975).
40. Chen, Y. C., et al. Generation and characterization of monoclonal antibodies against dengue virus type 1 for epitope mapping and serological detection by epitope-based peptide antigens. Clin Vaccine Immunol 14, 404-411 (2007).
41. Liu, I. J., Chiu, C. Y., Chen, Y. C. & Wu, H. C. Molecular mimicry of human endothelial cell antigen by autoantibodies to nonstructural protein 1 of dengue virus. J Biol Chem 286, 9726-9736 (2011).
42. Tung, K. H., et al. CHC promotes tumor growth and angiogenesis through regulation of HIF-1alpha and VEGF signaling. Cancer Lett 331, 58-67 (2013).
43. Kao, S. H., et al. GSK3beta controls epithelial-mesenchymal transition and tumor metastasis by CHIP-mediated degradation of Slug. Oncogene 33, 3172-3182 (2014).
44. Koraishy, F. M., Silva, C., Mason, S., Wu, D. & Cantley, L. G. Hepatocyte growth factor (Hgf) stimulates low density lipoprotein receptor-related protein (Lrp) 5/6 phosphorylation and promotes canonical Wnt signaling. J Biol Chem 289, 14341-14350 (2014).
45. Hazan, R. B., Qiao, R., Keren, R., Badano, I. & Suyama, K. Cadherin switch in tumor progression. Ann N Y Acad Sci 1014, 155-163 (2004).
46. Shih, J. Y. & Yang, P. C. The EMT regulator slug and lung carcinogenesis. Carcinogenesis 32, 1299-1304 (2011).
47. Shih, J. Y., et al. Transcription repressor slug promotes carcinoma invasion and predicts outcome of patients with lung adenocarcinoma. Clin Cancer Res 11, 8070-8078 (2005).
48. Chang, T. H., et al. Slug confers resistance to the epidermal growth factor receptor tyrosine kinase inhibitor. Am J Respir Crit Care Med 183, 1071-1079 (2011).

1. An antibody or antigen-binding fragment thereof that specifically binds enolase 1 (ENO1), comprising
(a) a heavy chain variable region (VH) which comprises a heavy chain complementary determining region 1 (HC CDR1) comprising the amino acid sequence of SEQ ID NO: 2, a heavy chain complementary determining region 2 (HC CDR2) comprising the amino acid sequence of SEQ ID NO: 4, and a heavy chain complementary determining region 3 (HC CDR3) comprising the amino acid sequence of SEQ ID NO: 6; and
(b) a light chain variable region (VL) which comprises a light chain complementary determining region 1 (LC CDR1) comprising the amino acid sequence of SEQ ID NO: 9, a light chain complementary determining region (LC CDR2) comprising the amino acid sequence of SEQ ID NO: 11, and a light chain complementary determining region 3 (LC CDR3) comprising the amino acid sequence of SEQ ID NO: 13.

2. The antibody or antigen-binding fragment of claim 1, wherein the VH comprises the amino acid sequence of SEQ ID NO: 15.

3. The antibody or antigen-binding fragment of claim 1, wherein the VL comprises the amino acid sequence of SEQ ID NO: 16.

4. A recombinant nucleic acid comprising a nucleotide sequence encoding an antibody or antigen-binding fragment thereof as defined in claim 1.

5. A host cell comprising the recombinant nucleic acid of claim 4.

6. A composition, comprising an antibody or antigen-binding fragment thereof as defined in claim 1 and a carrier.

7. A method for inhibiting enolase 1 (ENO1)-downstream signaling and/or treating a disease or disorder associated with activation of ENO1-downstream signaling comprising
administering to a subject in need thereof an effective amount of an antibody or antigen-binding fragment thereof as defined in claim 1.

8. The method of claim 7, wherein the disease or disorder is a cancer and a metastasis thereof.

9. The method of claim 8, wherein the cancer is selected from the group consisting of lung cancer, brain cancer, breast cancer, cervical cancer, colon cancer, gastric cancer, head and neck cancer, kidney cancer, leukemia, liver cancer, ovarian cancer, pancreatic cancer, prostate cancer, skin cancer and testicular cancer.

10. The method of claim 9, wherein the cancer is lung cancer.

11. The method of claim 8, wherein the antibody or antigen-binding fragment thereof is administered in an amount effective to inhibit tumor growth and metastasis in the subject in need thereof.

12. The method of claim 7, wherein the antibody or antigen-binding fragment thereof is administered in an amount effective to prolong survival time in the subject in need thereof.

13. A method for detecting enolase 1 (ENO1), comprising
(i) contacting an antibody or antigen-binding fragment thereof as defined in claim 1 with a cell of a subject in need; and
(ii) detecting binding of the antibody or antigen-binding fragment thereof to the cell.

14. The method of claim 13, wherein the cell is present in a biological sample obtained from the subject, or the cell is present in the subject.

15. The method of claim 13, wherein the subject is at risk for, or suspected of having a cancer.

16. The method of claim 13, wherein the subject is afflicted with a cancer.

17. The method of claim 16, further comprising comparing the results of the detection with a reference level and determining prognosis for the subject based on the results of the comparison, wherein an elevated level of ENO1 is indicative of a negative prognosis.

18. The method of claim 17, wherein the negative prognosis is selected from the group consisting of a reduced survival rate, an increased tumor growth, an increased risk of metastasis, an increased risk of relapse, and any combination thereof.

19. The method of claim 15, wherein the cancer is selected from the group consisting of lung cancer, brain cancer, breast cancer, cervical cancer, colon cancer, gastric cancer, head and neck cancer, kidney cancer, leukemia, liver cancer, ovarian cancer, pancreatic cancer, prostate cancer, skin cancer and testicular cancer.

20. The method of claim 19, wherein the cancer is lung cancer.

21. A method for monitoring cancer progression in a patient afflicted with cancer comprising
(i) providing a first biological sample from the patient at a first time point;
(ii) providing a second biological sample from the patient at a second time point, which is later than the first time point;
(iii) detecting the levels of enolase 1 (ENO1) in the first and second biological samples, wherein the detection is performed by an immunoassay with an antibody or antigen-binding fragment thereof as defined in claim 1; and
(iv) determining cancer progression in the patient based on the levels of ENO1 in the first and second biological samples, wherein an elevated level of ENO1 in the second biological sample as compared to that in the first biological sample is indicative of cancer progression.

22. The method of claim 21, wherein the cancer is selected from the group consisting of lung cancer, brain cancer, breast cancer, cervical cancer, colon cancer, gastric cancer, head and neck cancer, kidney cancer, leukemia, liver cancer, ovarian cancer, pancreatic cancer, prostate cancer, skin cancer and testicular cancer.

23. The method of claim 22, wherein the cancer is lung cancer.

24.-28. (canceled)

29. A kit for performing a method of claim 13, which comprises
an antibody or antigen-binding fragment thereof that specifically binds enolase 1 (ENO1), comprising
(a) a heavy chain variable region (VH) which comprises a heavy chain complementary determining region 1 (HC CDR1) comprising the amino acid sequence of SEQ ID NO: 2, a heavy chain complementary determining region 2 (HC CDR2) comprising the amino acid sequence of SEQ ID NO: 4, and a heavy chain complementary determining region 3 (HC CDR3) comprising the amino acid sequence of SEQ ID NO: 6; and
(b) a light chain variable region (VL) which comprises a light chain complementary determining region 1 (LC CDR1) comprising the amino acid sequence of SEQ ID NO: 9, a light chain complementary determining region (LC CDR2) comprising the amino acid sequence of SEQ ID NO: 11, and a light chain complementary determining region 3 (LC CDR3) comprising the amino acid sequence of SEQ ID NO: 13; and
instructions for using the kit to detect ENO1.

30. A method for screening for a candidate agent for cancer therapy, comprising
(i) contacting an agent with a cancer cell expressing ENO1 on the cell surface of the cancer cell;
(ii) detecting binding of the agent with ENO1 on the cell surface of the cancer cell and preferably further measuring the levels of one or more ENO1-downstream signaling events/components in the cancer cell; and
(iii) determining if the agent is a candidate anti-cancer based on the results of the detection and/or measurement.

31. The method of claim 30, wherein the results of the detection showing that the agent binds to ENO1 on the cell surface indicates that the agent has an anti-cancer effect.

32. The method of claim 30, wherein the results of the measurement showing a decreased level of one or more of the ENO1-downstream signaling events/components in the cancer cell indicates that the agent has an anti-cancer effect.

33. The method of claim 30, wherein the one or more ENO1-downstream signaling events/components comprises HGF-triggered phosphorylation of HGFR, association between ENO1 and HGFR, and/or SLUG steady-state protein.

34. The method of claim 30, wherein the anti-cancer effect comprises inhibition of tumor growth and/or metastasis.

35. The method of claim 30, wherein the cancer is selected from the group consisting of lung cancer, brain cancer, breast cancer, cervical cancer, colon cancer, gastric cancer, head and neck cancer, kidney cancer, leukemia, liver cancer, ovarian cancer, pancreatic cancer, prostate cancer, skin cancer and testicular cancer.

36. The method of claim 35, wherein the cancer is lung cancer.


SEQUENCE LISTING (16 sequences)

SEQ ID NO: 1 - protein, 30 aa, Mus musculus
QVQLQQSGPEVVRPGVSVKISCKGSGYTFT

SEQ ID NO: 2 - protein, 5 aa, Mus musculus
DYAMH

SEQ ID NO: 3 - protein, 14 aa, Mus musculus
WVKQSHAKSLEWIG

SEQ ID NO: 4 - protein, 17 aa, Mus musculus
VISTYNGNTNYNQKFKG

SEQ ID NO: 5 - protein, 32 aa, Mus musculus
KATMTVDKSSSTAYMELARLTSEDSAIYYCAR

SEQ ID NO: 6 - protein, 5 aa, Mus musculus
SPLRY

SEQ ID NO: 7 - protein, 11 aa, Mus musculus
WGQGTTLTVSS

SEQ ID NO: 8 - protein, 23 aa, Mus musculus
QIVLSQSPAILSASPGEKVTMTC

SEQ ID NO: 9 - protein, 10 aa, Mus musculus
RASSSVSYMH

SEQ ID NO: 10 - protein, 15 aa, Mus musculus
WYQQKPGSSPKPWIY

SEQ ID NO: 11 - protein, 7 aa, Mus musculus
ATSNLAS

SEQ ID NO: 12 - protein, 32 aa, Mus musculus
GVPARFSGSGSGTSYSLTISRVEAEDAATYYC

SEQ ID NO: 13 - protein, 9 aa, Mus musculus
QQWSSNPLT

SEQ ID NO: 14 - protein, 10 aa, Mus musculus
FGAGTKLELK

SEQ ID NO: 15 - protein, 114 aa, Mus musculus
QVQLQQSGPEVVRPGVSVKISCKGSGYTFTDYAMHWVKQSHAKSLEWIGVISTYNGNTNYNQKFKGKATMTVDKSSSTAYMELARLTSEDSAIYYCARSPLRYWGQGTTLTVSS

SEQ ID NO: 16 - protein, 106 aa, Mus musculus
QIVLSQSPAILSASPGEKVTMTCRASSSVSYMHWYQQKPGSSPKPWIYATSNLASGVPARFSGSGSGTSYSLTISRVEAEDAATYYCQQWSSNPLTFGAGTKLELK